(19) United States
(12) Patent Application Publication
(10) Pub. No.: US 2023/0392188 A1
(43) Pub. Date: Dec. 7, 2023

(54) ELECTROPHORESIS-MEDIATED CHARACTERIZATION OF DNA CONTENT OF ADENO-ASSOCIATED VIRUS CAPSIDS

(71) Applicant: PerkinElmer Health Sciences, Inc., Waltham, MA (US)

(72) Inventors: James White, Waltham, MA (US); Brendan Curran, Waltham, MA (US); Lloyd Bwanali, Waltham, MA (US); James Atwood, Waltham, MA (US); Shreyas Shah, Waltham, MA (US); Dipti Mehta, Waltham, MA (US); Menel Ben Frej, Waltham, MA (US)

(21) Appl. No.: 18/204,037

(22) Filed: May 31, 2023

Related U.S. Application Data
(60) Provisional application No. 63/348,166, filed on Jun. 2, 2022.

(51) Int. Cl. C12Q 1/6806; C12N 15/87; C12Q 1/6816

(52) U.S. Cl. CPC C12Q 1/6806; C12N 15/87; C12Q 1/6816

(57) ABSTRACT

According to aspects of methods of characterizing a population of particles putatively containing recombinant nucleic acids in a fluid sample of the present disclosure, includes extracting nucleic acids from the particles, labeling the extracted recombinant nucleic acids, separating the labeled nucleic acids by size, and comparing the separated labeled nucleic acids with a standard to determine one or more of: 1) a ratio of full viral particles to empty viral particles in the fluid sample, 2) a ratio of full viral particles or empty viral particles to partially full viral particles, and 3) a ratio of viral particles containing an intact recombinant genome to viral particles containing an incomplete recombinant genome, thereby characterizing the population of particles in the fluid sample. Optionally included are extracting proteins from the particles, labeling the extracted proteins, separating the labeled proteins according to size, and comparing the separated labeled proteins with a standard.

### CROSS-REFERENCE TO RELATED APPLICATION

This application claims priority to U.S. Provisional Patent Application Ser. No. 63/348,166, filed Jun. 2, 2022, the entire content of which is incorporated herein by reference.

### FIELD OF THE INVENTION

The present disclosure relates generally to assessment of DNA content of a population of particles putatively containing recombinant nucleic acids. According to specific aspects, the present disclosure relates to assessment of a population of particles putatively containing recombinant nucleic acids to determine: 1) the proportion of particles in a sample that correctly contain intact recombinant nucleic acids (“full”), 2) the proportion of particles in a sample that contain recombinant nucleic acids of the incorrect size (“partially full”), 3) the proportion of viral capsids in a sample that contain no detectable recombinant nucleic acids (“empty”), or 4) any combination of 1), 2), and 3).

### BACKGROUND OF THE INVENTION

Particles containing recombinant nucleic acids, such as viral vectors, have various utilities, including use in recombinant technologies, and gene therapy. Among the current gene therapy, viral vectors, including adeno-associated virus (AAV), has been demonstrated to provide successful, long-term gene transfer in vivo.

AAV is a single-stranded DNA (ssDNA), non-enveloped virus, that belongs to the parvovirus family, genus Dependoparvovirus. AAV particles consist of an icosahedral capsid composed of three types of structural proteins, VP1, VP2, and VP3, and a single-stranded DNA genome. Numerous serotypes of naturally occurring and recombinant serotypes of AAV exist which specifically target particular tissues and cells, and AAV is able to infect both dividing and non-dividing cells.

However, processes of AAV production result in generation of mixtures of viral particles some of which correctly contain the desired recombinant DNA, also referred to as “full” capsids, some of which are capsids that only contain part of the desired recombinant DNA, also referred to as “partially full”, and some of which are capsids that have do not correctly contain DNA, also referred to as “empty” capsids.

Similarly, processes of production of non-virus particles which are carriers for recombinant nucleic acids, i.e. a non-virus carrier particle, result in generation of mixtures of non-virus carrier particles some of which correctly contain the desired recombinant DNA, also referred to as “full” non-virus carrier particles, some of which are only contain part of the desired recombinant DNA, also referred to as “partially full”, and some of which are non-virus carrier particles that do not correctly contain DNA, also referred to as “empty” non-virus carrier particles.

There is a continuing need for methods of characterizing a population of particles putatively containing recombinant nucleic acids in a sample to determine a ratio of particles in the sample correctly containing intact recombinant nucleic acids i.e. “full”, to particles in the sample which do not correctly contain intact recombinant nucleic acids, i.e. “empty”, and/or “partially full”.

### SUMMARY OF THE INVENTION

Methods of characterizing a population of particles putatively containing recombinant nucleic acids in a fluid sample to determine one or more of: 1) a ratio of full particles to empty particles in the fluid sample, and 2) a ratio of full particles or empty particles to partially full particles, comprising: extracting the recombinant nucleic acids from a first aliquot of the fluid sample containing the particles, producing extracted recombinant nucleic acids; labeling the extracted recombinant nucleic acids, producing labeled extracted recombinant nucleic acids; flowing the labeled extracted recombinant nucleic acids through a polymeric separation medium in the microchannel into a detection region in fluid communication with the microchannel, the detection region in signal communication with a sensor capable of detecting a signal from the detectable nucleic acid label of the labeled extracted recombinant nucleic acids, whereby the labeled extracted recombinant nucleic acids are separated according to size by flowing the labeled extracted recombinant nucleic acids through the polymeric separation medium of the microchannel; detecting the detectable label of the labeled extracted recombinant nucleic acids in the detection region to determine: a) an amount of time taken by the labeled extracted recombinant nucleic acids to flow through the polymeric separation medium in the microchannel into the detection region, indicative of size of the recombinant nucleic acids in the fluid sample, and/or b) strength of the signal of the detectable label in the detection region, representative of the amount of labeled extracted recombinant nucleic acids present, and indicative of concentration of the labeled extracted recombinant nucleic acids in the fluid sample; and comparing a) to a reference standard representative of full particles and, based on the comparison, determining a ratio of full particles to partially full particles, producing a first assay result and/or comparing b) to a reference standard representative of full particles and, based on the comparison, determining a ratio of full particles to empty particles in the fluid sample, producing a first assay result; thereby characterizing the population of particles in the fluid sample.

Methods of characterizing a population of virus particles putatively containing recombinant DNA in a fluid sample to determine one or more of: 1) a ratio of full virus particles to empty virus particles in the fluid sample, and 2) a ratio of full virus particles or empty virus particles to partially full virus particles, comprising: extracting the recombinant DNA from a first aliquot of the fluid sample containing the virus particles, producing extracted recombinant DNA, if present; labeling the extracted recombinant DNA, producing labeled extracted recombinant DNA; flowing the labeled extracted recombinant DNA through a polymeric separation medium in the microchannel into a detection region in fluid communication with the microchannel, the detection region in signal communication with a sensor capable of detecting a signal from the detectable label of the labeled extracted recombinant DNA, whereby the labeled extracted recombinant DNA is separated according to size by flowing the labeled extracted recombinant DNA through the polymeric separation medium of the microchannel; detecting the detectable label of the labeled extracted recombinant DNA in the detection region to determine: a) an amount of time taken by the labeled extracted recombinant DNA to flow through the polymeric separation medium in the microchannel into the detection region, indicative of size of the recombinant DNA in the fluid sample, and/or b) strength of the signal of the detectable label in the detection region, representative of the amount of labeled extracted recombinant DNA present, and indicative of concentration of the labeled extracted recombinant DNA in the fluid sample; and comparing a) to a reference standard representative of full virus particles and, based on the comparison, determining a ratio of full virus particles to partially full virus particles, producing a first assay result (RDNA) and/or comparing b) to a reference standard representative of full virus particles and, based on the comparison, determining a ratio of full virus particles to empty virus particles in the fluid sample, producing a first assay result; thereby characterizing the population of virus particles in the fluid sample.

According to aspects of methods of characterizing a population of particles putatively containing recombinant nucleic acids in a fluid sample of the present disclosure, labeling the extracted recombinant nucleic acids comprises introducing the extracted recombinant nucleic acids into a well and/or microchannel of a microfluidic device, the well and/or microchannel comprising a polymeric separation medium and a detectable nucleic acid label, whereby the detectable nucleic acid label binds to the extracted recombinant nucleic acids, producing labeled extracted recombinant nucleic acids in the well and/or microchannel.

Methods of characterizing a population of particles putatively containing recombinant nucleic acids in a fluid sample according to aspects of the present disclosure, further include: determining an amount of particle protein present in the fluid sample.

According to aspects of methods of characterizing a population of particles putatively containing recombinant nucleic acids in a fluid sample of the present disclosure, the amount of particle protein in the fluid sample is determined using information about a total number of particles in the fluid sample without assaying the particle protein in the sample.

Methods of characterizing a population of particles putatively containing recombinant nucleic acids in a fluid sample according to aspects of the present disclosure, further include: assaying particle protein in a second aliquot of the fluid sample to determine an amount of particle protein present in the fluid sample.

According to aspects of methods of characterizing a population of particles putatively containing recombinant nucleic acids in a fluid sample of the present disclosure, assaying particle protein in a second aliquot of the fluid sample to determine an amount of particle protein present in the fluid sample includes: extracting protein from a second aliquot of the fluid sample containing the particles, producing extracted proteins of the particles; labeling the extracted proteins of the particles, producing labeled extracted proteins of the particles;

flowing the labeled extracted proteins of the particles through the polymeric separation medium in the microchannel into a detection region in fluid communication with the microchannel, the detection region in signal communication with a sensor capable of detecting a signal from the detectable label of the labeled extracted proteins, whereby the labeled extracted proteins are separated according to size by flowing the extracted proteins through the polymeric separation medium of the microchannel; detecting the detectable label of the labeled extracted proteins to determine: c) an amount of time taken by the labeled extracted proteins to flow through the polymeric separation medium in the microchannel into the detection region, and d) strength of the signal of the detectable label representative of the amount of labeled extracted proteins present; comparing c) and d) to a reference standard, the reference standard representing a known amount of the protein, thereby determining a relative amount of protein in the sample, and thereby producing a second assay result (Rₚ); and comparing the first assay result and the second assay result, thereby determining one or more of: 1) a ratio of full viral particles to empty viral particles in the fluid sample, 2) a ratio of full viral particles or empty viral particles to partially full viral particles, and 3) a ratio of viral particles containing an intact recombinant genome to viral particles containing an incomplete recombinant genome, thereby characterizing the population of recombinant viral particles in the fluid sample.

According to aspects of methods of characterizing a population of particles putatively containing recombinant nucleic acids in a fluid sample of the present disclosure, labeling the extracted proteins of the particles comprises introducing the extracted proteins of the particles into a well and/or microchannel of a microfluidic device, the well and/or microchannel comprising a polymeric separation medium and a detectable protein label, whereby the detectable protein label binds to the extracted proteins of the particles, producing labeled extracted proteins of the particles in the well and/or microchannel.

According to aspects of methods of characterizing a population of particles putatively containing recombinant nucleic acids in a fluid sample of the present disclosure, the particles are recombinant virus particles having a single-stranded DNA or RNA genome in the range of 500-7000 nucleotides in length.

According to aspects of methods of characterizing a population of particles putatively containing recombinant nucleic acids in a fluid sample of the present disclosure, the particles are selected from the group consisting of: recombinant adeno-associated virus (AAV) particles, recombinant retrovirus particles, recombinant lentivirus particles, and recombinant adenovirus particles.

According to aspects of methods of characterizing a population of particles putatively containing recombinant nucleic acids in a fluid sample of the present disclosure, the particles are recombinant virus particles in the fluid sample present in an amount of about 1×10¹⁰ recombinant viral particles/milliliter to about 1×10¹⁴ recombinant viral particles/milliliter.

According to aspects of methods of characterizing a population of particles putatively containing recombinant nucleic acids in a fluid sample of the present disclosure, extracting nucleic acids from a first aliquot of the fluid sample containing particles comprises contacting the first aliquot of the fluid sample containing particles with a proteinase and a denaturing agent. According to aspects of methods of characterizing a population of particles putatively containing recombinant nucleic acids in a fluid sample of the present disclosure, the denaturing agent is heat, a chaotropic agent, a detergent, or a mixture thereof.

According to aspects of methods of characterizing a population of particles putatively containing recombinant nucleic acids in a fluid sample of the present disclosure, extracting nucleic acids from a first aliquot of the fluid sample containing particles comprises contacting the first aliquot of the fluid sample containing particles with a serine proteinase and a denaturing agent. According to aspects of methods of characterizing a population of particles putatively containing recombinant nucleic acids in a fluid sample of the present disclosure, the denaturing agent is a chaotropic agent, a detergent, or a mixture thereof.

According to aspects of methods of characterizing a population of particles putatively containing recombinant nucleic acids in a fluid sample of the present disclosure, extracting nucleic acids from a first aliquot of the fluid sample containing particles comprises contacting the first aliquot of the fluid sample containing particles with a proteinase and a denaturing agent, wherein the proteinase is selected from the group consisting of: proteinase K, subtilisin, trypsin, chymotrypsin, thrombin, plasmin, elastase, pronase, and lactoferrin. According to aspects of methods of characterizing a population of particles putatively containing recombinant nucleic acids in a fluid sample of the present disclosure, the denaturing agent is heat, a chaotropic agent, a detergent, or a mixture thereof.

Methods of characterizing a population of virus particles putatively containing recombinant nucleic acids in a fluid sample to determine one or more of: 1) a ratio of full virus particles (N_{f}) to empty virus particles (Nₑ) in the fluid sample, 2) a ratio of full virus particles or empty virus particles to partially full virus particles according to aspects of the present disclosure include: extracting viral nucleic acids from a first aliquot of the fluid sample; assaying the extracted viral nucleic acids by microfluidic electrophoretic assay; calculating a ratio of relative nucleic acid concentration in the fluid sample to nucleic concentration in a reference standard, calculating a ratio of relative protein concentration in the fluid sample to protein concentration in a reference standard, and calculating a fractional amount of full capsids, β, in the sample according to:

$$β = \frac{N ( f )}{N ( f ) + N ( e )} = \frac{{β}_{s} {R}_{DNA}}{{R}_{p}} , \qquad ( 6 )$$

wherein N is a total number of capsids, f and e refer to full and empty, respectively, βs is the percent of full capsids in the reference standard, R_{DNA} is the ratio of concentration of viral nucleic acids in the sample to the concentration of viral nucleic acids in the reference standard, and R_{P} is the ratio of concentration of viral proteins in the sample to the concentration of viral proteins in the reference standard.

Methods of characterizing a population of virus particles putatively containing recombinant nucleic acids in a fluid sample to determine one or more of: 1) a ratio of full virus particles (N_{f}) to empty virus particles (Nₑ) in the fluid sample, 2) a ratio of full virus particles or empty virus particles to partially full virus particles according to aspects of the present disclosure include: extracting viral nucleic acids from a first aliquot of the fluid sample; assaying the extracted viral nucleic acids by microfluidic electrophoretic assay; extracting viral proteins from a second aliquot of the fluid sample; assaying at least one viral protein of the extracted viral proteins by microfluidic electrophoretic assay; calculating a ratio of nucleic acid concentration in the fluid sample to nucleic concentration in a reference standard, calculating a ratio of protein concentration in the fluid sample to protein concentration in a reference standard, and calculating a fractional amount of full capsids, β, in the sample according to:

$$β = \frac{N ( f )}{N ( f ) + N ( e )} = \frac{{β}_{s} {R}_{DNA}}{{R}_{p}} , \qquad ( 6 )$$

wherein N is a total number of capsids, f and e refer to full and empty, respectively, βs is the percent of full capsids in the reference standard, R_{DNA} is the ratio of concentration of viral nucleic acids in the sample to the concentration of viral nucleic acids in the reference standard, and R_{P} is the ratio of concentration of viral proteins in the sample to the concentration of viral proteins in the reference standard.

Methods of characterizing a population of virus particles putatively containing recombinant nucleic acids in a fluid sample to determine one or more of: 1) a ratio of full virus particles (N_{f}) to empty virus particles (Nₑ) in the fluid sample, 2) a ratio of full virus particles or empty virus particles to partially full virus particles according to aspects of the present disclosure include: extracting viral nucleic acids from a first aliquot of the fluid sample; assaying the extracted viral nucleic acids by microfluidic electrophoretic assay; calculating a ratio of nucleic acid concentration in the fluid sample to nucleic concentration in a reference standard, calculating a ratio of protein concentration in the fluid sample to protein concentration in a reference standard, wherein protein concentration in the fluid sample is calculated by reference to a standard and no protein assay is performed, and calculating a fractional amount of full capsids, (3, in the sample according to:

$$β = \frac{N ( f )}{N ( f ) + N ( e )} = \frac{{β}_{s} {R}_{DNA}}{{R}_{p}} , \qquad ( 6 )$$

wherein N is a total number of capsids, f and e refer to full and empty, respectively, βs is the percent of full capsids in the reference standard, R_{DNA} is the ratio of concentration of viral nucleic acids in the sample to the concentration of viral nucleic acids in the reference standard, and R_{P} is the ratio of concentration of viral proteins in the sample to the concentration of viral proteins in the reference standard.

Kits for characterizing nucleic acid content of a population of particles putatively containing recombinant nucleic acids in a fluid sample are provided according to aspects of the present disclosure, which include: a nucleic acid label, a gel, a nucleic acid ladder standard, a nucleic acid storage buffer, a nucleic acid sample buffer, a proteinase, and a denaturing agent. According to aspects of the present disclosure, the nucleic acid label is a fluorescent nucleic acid intercalator. According to aspects of the present disclosure, the proteinase is a serine protease.

Kits for characterizing nucleic acid content of a population of particles putatively containing recombinant nucleic acids in a fluid sample are provided according to aspects of the present disclosure, which include: a nucleic acid label, a gel, a nucleic acid ladder standard, a nucleic acid storage buffer, a nucleic acid sample buffer, a proteinase, a denaturing agent, a protein dye, a protein standard, a protein storage buffer, a protein sample buffer, and a wash buffer. According to aspects of the present disclosure, the nucleic acid label is a fluorescent nucleic acid intercalator. According to aspects of the present disclosure, the proteinase is a serine protease.

Kits for characterizing nucleic acid content of a population of virus particles putatively containing recombinant nucleic acids in a fluid sample are provided according to aspects of the present disclosure, which include: a nucleic acid label, a gel, a nucleic acid ladder standard, a nucleic acid storage buffer, a nucleic acid sample buffer, a proteinase, and a denaturing agent. According to aspects of the present disclosure, the nucleic acid label is a fluorescent nucleic acid intercalator. According to aspects of the present disclosure, the proteinase is a serine protease. According to aspects of the present disclosure, the virus particles are AAV virus particles and an AAV standard containing a known number of AAV virus particles is included in a kit for characterizing nucleic acid content of a population of AAV particles putatively containing recombinant nucleic acids in a fluid sample.

Kits for characterizing nucleic acid content of a population of virus particles putatively containing recombinant nucleic acids in a fluid sample are provided according to aspects of the present disclosure, which include: a nucleic acid label, a gel, a nucleic acid ladder standard, a nucleic acid storage buffer, a nucleic acid sample buffer, a proteinase, a denaturing agent, a protein dye, a protein standard, a protein storage buffer, a protein sample buffer, and a wash buffer. According to aspects of the present disclosure, the nucleic acid label is a fluorescent nucleic acid intercalator. According to aspects of the present disclosure, the proteinase is a serine protease. According to aspects of the present disclosure, the protein standard is or includes a protein ladder standard. According to aspects of the present disclosure, the virus particles are AAV virus particles and an AAV standard containing a known number of AAV virus particles is included in a kit for characterizing content of a population of AAV particles putatively containing recombinant nucleic acids in a fluid sample.

Kits for characterizing nucleic acid content of a population of particles putatively containing recombinant nucleic acids in a fluid sample are provided according to aspects of the present disclosure, wherein the proteinase is selected from the group consisting of: proteinase K, subtilisin, trypsin, chymotrypsin, thrombin, plasmin, elastase, pronase, and lactoferrin.

Kits for characterizing nucleic acid content of a population of particles putatively containing recombinant nucleic acids in a fluid sample are provided according to aspects of the present disclosure, wherein the denaturing agent is a chaotropic agent, a detergent, or a mixture thereof.

Kits for characterizing nucleic acid content of a population of particles putatively containing recombinant nucleic acids in a fluid sample are provided according to aspects of the present disclosure contain at least one proteinase and at least one chaotropic agent, wherein the at least one proteinase is proteinase K and the at least one chaotropic agent is urea.

Kits for characterizing nucleic acid content of a population of particles putatively containing recombinant nucleic acids in a fluid sample are provided according to aspects of the present disclosure include one or more standards. According to aspects of the present disclosure, an included standard may be a sample including a known ratio of full particles:empty particles, a known ratio of full particles:partially full particles, or a known ratio of empty particles:partially full particles, two or more thereof, or all three thereof. According to aspects of the present disclosure, an included standard may be a sample including a known ratio of full AAV particles:empty AAV particles, a known ratio of full AAV particles:partially full AAV particles, or a known ratio of empty AAV particles:partially full AAV particles, two or more thereof, or all three thereof.

### BRIEF DESCRIPTION OF THE DRAWINGS

FIG. 1 is a diagram showing aspects of a method of characterizing a population of particles putatively containing recombinant nucleic acids in a fluid sample to determine one or more of: 1) a ratio of full particles to empty particles in the fluid sample, 2) a ratio of full particles or empty particles to partially full particles, according to the present disclosure; in the example shown, the method achieves viral capsid nucleic acid and protein content analysis;

FIG. 2 is a diagram showing aspects of a method of characterizing a population of particles putatively containing recombinant nucleic acids in a fluid sample to determine one or more of: 1) a ratio of full particles to empty particles in the fluid sample, 2) a ratio of full particles or empty particles to partially full particles, according to the present disclosure; in the example shown, the method achieves viral capsid nucleic acid and protein content analysis;

FIG. 3A is a typical protein electropherogram showing peaks representative of VP3, VP2 and VP1 AAV capsid proteins, from smallest to largest weight (left to right) from samples containing either full or empty AAV capsids;

FIG. 3B is a graph of the results shown in FIG. 3A showing the summarized area under the curve (“area”) for each of the three main protein peaks for VP1, VP2, and VP3, respectively, demonstration no statistical difference between the VP peaks yielded by the full virus capsids and empty virus capsids;

FIG. 4A is a typical nucleic acid electropherogram showing results obtained using samples containing either full or empty AAV capsids; note that the result using a sample containing full AAV capsids produces a peak, while the result using a sample containing empty AAV capsids does not show a peak;

FIG. 4B is a graph of the results shown in FIG. 4A showing the summarized area under the curve (“area”) for each of the samples containing either full or empty AAV capsids; note that for all three runs the empty AAV sample did not produce a peak, showing a significant statistical difference between the ssDNA profiles of the empty vs full samples;

FIG. 5A is a typical electropherogram showing capsid protein results obtained using samples containing different percentages of full capsids, including 75%, 50%, 25%, and 4% full capsids; the electropherogram in this figure shows peaks representative of VP3, VP2 and VP1 capsid proteins, from smallest to largest weight (left to right) for all of the samples;

FIG. 5B is a graph of the results shown in FIG. 5A showing the summarized area under the curve (“area”) of the VP protein peaks for the four samples containing the indicated varying full capsid percentages;

FIG. 5C is a typical nucleic acid electropherogram showing nucleic acid results obtained using samples containing different percentages of full capsids, including 75%, 50%, 25%, and 4% full capsids; note that the DNA peak is observed to decrease as the percentage of full capsids decreases;

FIG. 5D is a graph of the results shown in FIG. 5C showing the summarized area under the curve (“area”) of the virus nucleic acid peaks for the four samples containing the indicated varying full capsid percentages; note that for all three runs the empty AAV sample did not produce a peak;

FIG. 6 is a graph showing prediction accuracy between the protein area prediction method based on Equation (6) and the concentration prediction method based on Equation (8) in comparison to the actual or reported percentage of full capsids of each sample; the sample numbers correspond to those described in Tables 6-8; Moreover, all samples include an error bar of 10% (in each direction) of the total value to account for inaccuracies in the reported standards used to prepare both the standard and samples used in this study;

FIG. 7 is a protein electropherogram showing integration of the VP3 peak for Empty/Full analysis of AAV protein from samples containing empty or full AAV capsids;

FIG. 8 is an electropherogram showing integration of ssDNA peak for Empty/Full analysis of AAV nucleic acid from samples containing empty or full AAV capsids;

FIG. 9 is a nucleic acid electropherogram showing integration of ssDNA peak for Partially Full/Full analysis of AAV nucleic acid from samples containing partially full capsids or full AAV capsids;

FIG. 10 is an electropherogram showing integration of RNA peaks of an RNA ladder without (top line) or with (bottom line) benzonase treatment;

FIG. 11 is an overlay electropherogram of 3 sips of AAV8 ssDNA obtained after recovery from a clean-up procedure using a ssDNA purification kit (QIAquick PCR);

FIG. 12A is an overlay electropherogram showing separation of two nucleic acid samples of sizes a 2.4 kilonucleotide (knt) SSDNA and a 3.3 knt SSDNA in separate capillary electrophoresis runs;

FIG. 12B is an overlay electropherogram showing separation of two nucleic acid samples of sizes a 2.4 kilo nucleotide (knt) SSDNA and a 3.3 knt SSDNA merged in a single capillary electrophoresis run;

FIG. 13A is a set of electropherograms showing extracted viral protein assay results for each serotype indicated, AAV2, AAV9, or AAV8, shown top to bottom. Viral protein resolution and full-width at half-maximum (FWHM) is similar for AAV2, AAV8, or AAV9 virus particles;

FIG. 13B is a graph showing extracted nucleic acids assay results for 70% full AAV8 virus particles (F-70), 50% full AAV8 virus particles (F-50), 25% full AAV8 virus particles (F-25), and empty AAV8 virus particles (F-0). For each pair of bars on the graph, the left-hand bar represents the theoretical percentage full as calculated, and the right-hand bar represents the average percentage full as measured and calculated using equation (6);

FIG. 13C is a graph showing extracted nucleic acids assay results for 82% full AAV9 virus particles (F-82), 50% full AAV9 virus particles (F-50), 25% full AAV9 virus particles (F-25), and empty AAV9 virus particles (F-0). For each pair of bars on the graph, the left-hand bar represents the theoretical percentage full as calculated, and the right-hand bar represents the average percentage full as measured and calculated using equation (6); and

FIG. 13D is a graph showing extracted nucleic acids assay results for 62% full AAV2 virus particles (F-62), 50% full AAV2 virus particles (F-50), 25% full AAV2 virus particles (F-25), and empty AAV2 virus particles (F-0). For each pair of bars on the graph, the left-hand bar represents the theoretical percentage full as calculated, and the right-hand bar represents the average percentage full as measured and calculated using equation (6).

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, PA: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

The terms “first,” “second,” and the like are used herein to describe various features or elements, but these features or elements are not intended to be limited by these terms, but are only used to distinguish one feature or element from another feature or element. Thus, a first feature or element could be termed a second feature or element, and vice versa, without departing from the teachings of the present disclosure.

Methods of characterizing a population of particles putatively containing recombinant nucleic acids in a fluid sample to determine one or more of: 1) a ratio of full particles to empty particles in the fluid sample, and 2) a ratio of full particles or empty particles to partially full particles, are provided according to aspects of the present disclosure.

The term “particle”, and grammatical equivalents thereof, as used herein, refers to: 1) a virus, i.e. a viral particle, or 2) to a non-virus particle which is a carrier for recombinant nucleic acids, i.e. a non-virus carrier particle.

The terms “viral particle” as used herein refers to a “complete” viral particle including a capsid and an encapsidated viral genome. For example, when referring to a recombinant adeno-associated virus (AAV) viral particle, the viral particle includes an adeno-associated virus capsid and an encapsidated recombinant genome.

The term “recombinant viral particle” as used herein refers to a viral capsid that contains a heterologous nucleic acid wherein the recombinant viral particle is generated by use of recombinant techniques and/or recombinant technology. The term “heterologous nucleic acid” as used herein refers to a nucleic acid that encodes a corresponding nucleic acid and/or polypeptide not encoded by the naturally occurring virus from which the recombinant viral particle is derived.

The recombinant nucleic acids putatively present in the population of particles to be analyzed may be any type of nucleic acids, and the term “nucleic acids” as used herein refers to RNA or DNA molecules having more than one nucleotide in any form including single-stranded, double-stranded, oligonucleotide or polynucleotide. According to aspects of the present disclosure, the particles are virus particles and the nucleic acids are single stranded RNA or DNA. According to aspects of the present disclosure, the particles are virus particles and the nucleic acids are single stranded DNA.

Examples herein describe methods according to aspects of the present disclosure with reference to Adeno-Associated Virus (AAV), and such methods may be used with other viruses including, without limitation, lentivirus, adenovirus, MMLV retrovirus, MSCV retrovirus, bacluovirus, vesicular stomatitis virus, and herpes simplex virus.

Subtypes of AAV were originally identified by serological methods resulting in identification of 12 canonical serotypes: AAV1, AAV2, AAV3, AAV4, AAV5, AAV6, AAV7, AAV8, AAV9, AAV10, AAV11, and AAV12. With the advent of molecular analytic techniques, numerous naturally occurring genetic variants have been identified and many synthetic genetic variants have been generated.

Methods according to aspects of the present disclosure are not limited with respect to analysis of a particular serotype or genetic variation of virus. In particular aspects, methods according to aspects of the present disclosure are not limited with respect to analysis of a particular serotype or genetic variation of AAV.

Non-virus particles which are carriers for recombinant nucleic acids, i.e. non-virus carrier particles, are capable of containing, or being associated with, recombinant nucleic acids, and may be used to transport the nucleic acids to a desired locations, for example into an organism, cell, or cell compartment.

Non-virus carrier particles may be, or include, liposomes, micelles, unilamellar or mulitlamellar vesicles; polymer particles such as hydrogel particles, polyglycolic acid particles or polylactic acid particles; inorganic particles; and inorganic/organic particles.

Non-virus carrier particles can be selected from among a lipid particle; a polymer particle; an inorganic particle; an inorganic/organic particle; or a mixture of any two or more thereof. A mixture of particle types can also be included as a particulate pharmaceutically acceptable carrier.

Non-virus carrier particles can be nanoparticles and/or microparticles. Non-virus carrier particles are typically formulated such that the particles have an average particle size in the range of about 1 nm-10 microns. In particular aspects of the present disclosure, non-virus carrier particles are formulated such that particles have an average particle size in the range of about 1 nm-100 nm.

Non-virus carrier particles include, for example, nanoparticulate polymers, dendrimers, liposomes, viruses, carbon nanotubes, and metals such as iron oxide and gold. Exemplary polymers for the preparation of non-virus carrier particles include natural polymers such as heparin, dextran, albumin, gelatin, alginate, collagen, and chitosan or synthetic polymers including polyethylene glycol (PEG), polyglutamic acid (PGA), polylactic acid (PLA), polycarprolactone (PCL) and N-(2-hydroxypropyl)-methacrylamide copolymer (HPMA).

Non-virus carrier particles are lipid-based carrier particles according to aspects of the present disclosure. The term “lipid-based carrier particles” refers to macromolecular structures having lipid and/or lipid derivatives as the major constituent.

Lipids included in lipid-based carrier particles can be naturally-occurring lipids, synthetic lipids or combinations thereof.

Lipid-based carrier particles are liposomes according to aspects of the present disclosure.

The term “liposome” refers to a bilayer particle of amphipathic lipid molecules enclosing an aqueous interior space. Liposomes are typically produced as small unilammellar vesicles (SUVs), large unilammellar vesicles (LUVs) or multilammellar vesicles (MLVs). Recombinant nucleic acids can be associated with liposomes by encapsulation in the aqueous interior space of the liposomes, disposed in the lipid bilayer of the liposomes and/or associated with the liposomes by binding, such as ionic binding or association by van der Waals forces. Liposomes according to aspects of the present disclosure are generally in the range of about 1 nanometer-1 micron in diameter although they are not limited with regard to size.

Lipid-based carrier particles include one or more types of neutral, cationic lipid and/or anionic lipid, such that the liposomal formulations have a surface charge or a net neutral surface charge at physiological pH. One or more PEG-modified lipids is optionally included.

The term cationic lipid refers to any lipid which has a net positive charge at physiological pH. Examples of cationic lipids include, but are not limited to, N-(1-(2,3-dioleyloxy)propyl)-N,N,N-trimethylammonium chloride (DOTMA); 1,2-dioleoyloxy-3-(trimethylammonium)propane (DOTAP); 1,2-dioleoyl-3-dimethylammonium-propane (DODAP); dioctadecylamidoglycylspermine (DOGS); 1,2-dipalmitoylphosphatidylethanolamidospermine (DPPES); 2,3-dioleyloxy-N-(2-(sp erminecarb oxami do)ethyl)-N,N-dim ethyl-1-prop anaminium trifluoroacetate (DOSPA); dimyristoyltrimethylammonium propane (DMTAP); (3-dimyristyloxypropyl)(dimethyl)(hydroxyethyl)ammonium (DMRIE); dioctadecyldimethylammonium chloride (DODAC), Dimethyldidodecylammonium bromide (DDAB); 3β[N-(N′,N′-dimethylaminoethane)-carbamoyl]cholesterol (DC-Chol); 1-[2-(9(Z)-octadecenoyloxy)-ethyl]-2-(8(Z)-heptadecenyl)-3-(2-hydroxyethyl)-imidazolinium (DOTIM); bis-guanidinium-spermidine-cholesterol (BGTC); bis-guanidinium-tren-cholesterol (BGTC); 1,3-Di-oleoyloxy-2-(6-carb oxy-spermyl)-propylamid (DO SPER) N-[3-[2-(1,3 -dioleoyloxy)propoxy-carbonyl]propyl]-N,N,N-trimethylammonium iodide (YKS-220); as well as pharmaceutically acceptable salts and mixtures thereof. Additional examples of cationic lipids are described in Lasic and Papahadjopoulos, Medical Applications of Liposomes, Elsevier, 1998; U.S. Pat. Nos. 4,897,355; 5,208,036; 5,264,618; 5,279,833; 5,283,185; 5,334,761; 5,459,127; 5,736,392; 5,753,613; 5,785,992; 6,376,248; 6,586,410; 6,733,777; and 7,145,039.

The term neutral lipid refers to any lipid which has no net charge, either uncharged or in neutral charge zwitterionic form, at physiological pH. Examples of neutral lipids include, but are not limited to, L-alpha-phosphatidylcholine (ePC), distearoylphosphatidylcholine (DSPC), dioleoylphosphatidylethanolamine (DOPE), di stearoylphosphatidylethanolamine (DSPE); 1,2-dioleoyl-sn-glycero-3-Phosphocholine (DOPC), 1,2-dipalmitoyl-sn-glycero-3-phosphoethanolamine (DPPE), cephalin, ceramide, cerebrosides, cholesterol, diacylglycerols, and sphingomyelin.

The term anionic lipid refers to any lipid which has a net negative charge at physiological pH. Examples of anionic lipids include, but are not limited to, dihexadecylphosphate (DhP), phosphatidyl inositols, phosphatidyl serines, such as dimyristoyl phosphatidyl serine, and dipalmitoyl phosphatidyl serine., phosphatidyl glycerols, such as dimyristoylphosphatidyl glycerol, dioleoylphosphatidyl glycerol, dilauryloylphosphatidyl glycerol, dipalmitoylphosphatidyl glycerol, di stearyloylphosphatidyl glycerol, phosphatidic acids, such as dimyristoyl phosphatic acid and dipalmitoyl phosphatic acid and diphosphatidyl glycerol.

The term “modified lipid” refers to lipids modified to aid in, for example, inhibiting aggregation and/or precipitation, inhibiting immune response and/or improving half-life in circulation in vivo. Modified lipids include, but are not limited to, pegylated lipids, such as polyethyleneglycol 2000 distearoylphosphatidylethanolamine (PEG(2000) DSPE); 1,2-dipalmitoyl-sn-glycero-3-phosphoethanolamine-N-[methoxy(polyethylene glycol)-2000] (DPPE-PEG-2000), and polyethyleneglycol 750 octadecylsphingosine (PEG(750) C8).

In addition to lipids, polymers, and inorganic materials, non-virus carrier particles may contain any of a variety of molecules and substances which may contribute to the structure of the particles and/or intended function of the particles and which are capable of being integrated into and/or complexed with the non-virus carrier particles including, but not limited to, proteins, peptides, carbohydrates, oligosaccharides, and drugs. Such molecules and substances may be biologically active molecules and substances. The term “biologically active molecules and substances” refers molecules or substances that exert a biological effect in vitro and/or in vivo, such as, but not limited to, nucleic acids, inhibitory RNA, siRNA, shRNA, ribozymes, antisense nucleic acids, antibodies, hormones, small molecules, aptamers, decoy molecules and toxins.

Non-virus carrier particles are generated using well-known standard methods, including, but not limited to, those described in detail in: Rangelov, S. et al. Polymer and Polymer-Hybrid Nanoparticles: From Synthesis to Biomedical Applications, CRC Press, 2013; Liposomes: A Practical Approach (The Practical Approach Series, 264), V. P. Torchilin and V. Weissig (Eds.), Oxford University Press; 2nd ed., 2003; N. Duzgunes, Liposomes, Part A, Volume 367 (Methods in Enzymology) Academic Press; 1st ed., 2003; L. V. Allen, Jr. et al., Ansel's Pharmaceutical Dosage Forms and Drug Delivery Systems, 8th Ed., Philadelphia, PA: Lippincott, Williams & Wilkins, 2005, pp. 663-666; and A. R. Gennaro, Remington: The Science and Practice of Pharmacy, Lippincott Williams & Wilkins, 21st ed., 2005, pp. 766-767.

The term “full” when used herein to refer to recombinant viral particles is intended to refer to viral capsids correctly containing an intact recombinant viral genome. The term “empty” as used herein to refer to recombinant viral particles is intended to refer to viral capsids which do not contain detectable nucleic acids. The term “partially full” as used herein to refer to recombinant viral particles is intended to refer to viral capsids containing an incomplete recombinant viral genome, i.e. not intact.

The term “full” when used herein to refer to non-virus carrier particles is intended to refer to non-virus carrier particles correctly containing an intact desired recombinant nucleic acid. The term “empty” as used herein to refer to non-virus carrier particles is intended to refer to non-virus carrier particles which do not contain detectable nucleic acids. The term “partially full” as used herein to refer to non-virus carrier particles is intended to refer to non-virus carrier particles containing an incomplete recombinant nucleic acid, i.e. not intact.

The term “incomplete recombinant viral genome” refers to a fragment of the recombinant genome, e.g. with one or more deleted ends, or a recombinant genome with one or more internal deletions. An incomplete recombinant genome is 5%, 10%, 15%, 20%, 25%, 30%, 35%, 40%, 45%, 50%, 55%, 60%, 65%, 70%, 75%, 80%, 85%, 90%, 95%, or more, or less, shorter in length than a reference complete recombinant viral genome.

The term “incomplete recombinant nucleic acid” refers to a fragment of the recombinant nucleic acid, e.g. with one or more deleted ends, or a recombinant nucleic acid with one or more internal deletions. An incomplete recombinant nucleic acid is 5%, 10%, 15%, 20%, 25%, 30%, 35%, 40%, 45%, 50%, 55%, 60%, 65%, 70%, 75%, 80%, 85%, 90%, 95%, or more, or less, shorter in length than a reference complete recombinant nucleic acid.

According to particular aspects of the present disclosure, methods of characterizing a population of particles in a sample to determine a ratio, or fractional percentage, of “full” particles to “empty” particles, and/or of “empty” particles to “full” particles, in the sample are provided.

According to specific aspects, the present disclosure relates to assessment of a population of recombinant viral capsids to determine: 1) the proportion of recombinant viral capsids in a sample that correctly contain an intact recombinant viral genome (“full”), 2) the proportion of recombinant viral capsids in a sample that contain a recombinant viral genome of the incorrect size (“partially full”), 3) the proportion of recombinant viral capsids in a sample that contain no detectable recombinant viral genome (“empty”), or 4) any combination of 1), 2), and 3).

According to particular aspects of the present disclosure, methods of characterizing a population of viral particles in a sample to determine a ratio, or fractional percentage, of 1) “partially full” viral capsids to “full” viral capsids, 2) “full” viral capsids to “partially full” viral capsids, 3) “partially full” viral capsids to “empty” viral capsids, 4) “empty” viral capsids to “partially full” viral capsids, or any combination of 1), 2), 3) and 4), in the sample are provided.

According to specific aspects, the present disclosure relates to assessment of a population of non-virus carrier particles to determine: 1) the proportion of non-virus carrier particles in a sample that correctly contain an intact recombinant nucleic acid (“full”), 2) the proportion of non-virus carrier particles in a sample that contain a recombinant nucleic acid of the incorrect size (“partially full”), 3) the proportion of non-virus carrier particles in a sample that contain no detectable recombinant nucleic acid (“empty”), or 4) any combination of 1), 2), and 3).

According to particular aspects of the present disclosure, methods of characterizing a population of non-virus carrier particles in a sample to determine a ratio, or fractional percentage, of 1) “partially full” non-virus carrier particles to “full” non-virus carrier particles, 2) “full” non-virus carrier particles to “partially full” non-virus carrier particles, 3) “partially full” non-virus carrier particles to “empty” non-virus carrier particles, 4) “empty” non-virus carrier particles to “partially full” non-virus carrier particles, or any combination of 1), 2), 3) and 4), in the sample are provided.

Methods of characterizing a population of particles putatively containing recombinant nucleic acids in a fluid sample to determine one or more of: 1) a ratio of full particles to empty particles in the fluid sample, and 2) a ratio of full particles or empty particles to partially full particles, are provided according to aspects of the present disclosure which include extracting the recombinant nucleic acids from a first aliquot of the fluid sample containing the particles, producing extracted recombinant nucleic acids. Further, and optionally, methods of characterizing a population of particles putatively containing recombinant nucleic acids in a fluid sample of the present disclosure, includes extracting protein from a second aliquot of the fluid sample containing the particles, producing extracted proteins of the particles.

Extracting the nucleic acids or proteins from the population of particles is accomplished by any method of separating nucleic acids or proteins from a sample containing a population of particles of interest.

Extraction of nucleic acids from a sample may include use of a protein denaturing agent effective to denature proteins and release the recombinant nucleic acids from the proteins in the sample. The protein denaturing agent can be, or include, one or more proteinases, heat, one or more detergents, osmotic shock, one or more chaotropic agents, one or more organic solvents, or a combination of any two or more thereof.

The denaturing agent can be, or include, a chaotropic agent, such as a thiocyanate salt such as guanidinium thiocyanate, sodium thiocyanate, potassium thiocyanate, or any combination of two or more thereof; n-butanol; ethanol; guanidinium chloride; lithium perchlorate; lithium acetate; magnesium chloride; phenol; 2-propanol; sodium dodecyl sulfate; lithium dodecyl sulfate; thiourea; formamide; urea; or a combination of any two or more thereof.

The denaturing agent can be, or include, a detergent. The detergent can be an anionic, cationic, zwitterionic, or non-ionic detergent, such as Triton X-100; Triton X-114; NP-40; Tween-20; Tween-80; octyl-beta-glucoside; octylthio glucoside; ethyl trimethyl ammonium bromide; sodium dodecyl sulfate (SDS); Brij-35; Brij-58; CHAPS; CHAPSO; or a combination of any two or more thereof

According to aspects of the present disclosure, the extraction of nucleic acids includes treatment of the particles with a proteinase. According to aspects of the present disclosure, treatment of the particles with a proteinase includes treatment of the particles with a serine protease. According to aspects of the present disclosure, treatment of the particles with a proteinase includes treatment of the particles with a proteinase selected from the group consisting of: proteinase K, subtilisin, trypsin, chymotrypsin, thrombin, plasmin, elastase, pronase, and lactoferrin.

According to aspects of the present disclosure, the extraction of nucleic acids includes treatment of the particles with a proteinase and a second denaturing agent. According to aspects of the present disclosure, the second denaturing agent for use in extracting nucleic acids from the particles is a chaotropic agent, a detergent, or a mixture thereof. According to aspects of the present disclosure, the second denaturing agent for use in extracting nucleic acids from the particles is, or includes, urea.

Extraction of proteins from a sample may include use of a nucleic acid denaturing agent effective to denature nucleic acids and release the proteins in the sample. The nucleic acid denaturing agent can be, or include, heating at a temperature of at least about 75° C.-100° C., and an optional reducing agent. According to aspects of the present disclosure, the optional reducing agent is, or includes, dithiotheitol.

Methods of characterizing a population of particles according to aspects of the present disclosure include labeling the extracted recombinant nucleic acids with a detectable label, producing labeled extracted recombinant nucleic acids.

Methods of characterizing a population of particles according to aspects of the present disclosure include labeling the extracted particle proteins with a detectable label, producing labeled extracted particle proteins.

The term “detectable label” refers to a material capable of producing a signal indicative of the presence of a labeled extracted recombinant nucleic acid or labeled extracted particle proteins and detectable by any appropriate method illustratively including spectroscopic, optical, photochemical, biochemical, enzymatic, electrical and/or immunochemical. A detectable label allows for detection based on detectable properties of the label, such as, but not limited to, chemical properties, electrical properties, magnetic properties, optical properties, physical properties, or any two or more thereof. The detectable label may include one or more of: a fluorescent label, a bioluminescent label, a chemiluminescent label, a chromophore, a magnetic label, an antibody, an antigen, an enzyme, a substrate, a radioisotope, or any two or more thereof

According to aspects of the present disclosure, the detectable label is a fluorescent label. A fluorescent label is selected based on fluorophore characteristics including, but not limited to, excitation maximum wavelength and emission maximum wavelength.

Fluorophores used as fluorescent labels can be any of numerous fluorophores including, but not limited to, those described in Haughland, R. P., The Handbook, A Guide to Fluorescent Probes and Labeling Technologies, 10th Ed., 2005; Lakowicz, J. R., Principles of Fluorescence Spectroscopy, Springer, 3rd ed., 2006; 4-acetamido-4′-isothiocyanatostilbene-2,2′disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcouluarin (Coumaran 151); cyanosine; DAPDXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′- isothiocyanatophenyl)-4-methylcoumarin; diethylenetriamine pentaacetate; 4,4′-diisothiocyanatodihydro-stilbene-2,2′-disulfonic acid; 4,4′-dii sothiocyanatostilb ene-2,2′-disulfonic acid; 5-[dimethylaminolnaphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin, 5-(4, 6-dichlorotriazin-2-yl)aminofluorescein (DTAF), 2′, 7′-dimethoxy-4′, 5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl}amino)naphthalene-1-sulfonic acid), Malachite Green isothiocyanate; 4-methylumbelliferone; orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron .RTM. Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid and terbium chelate derivatives, or any combination of two or more thereof.

According to aspects of the present disclosure, the detectable label is a fluorescent nucleic acid stain. Fluorescent nucleic acid stains include, but are not limited to, acridine dyes, acridine orange (AO, N,N,N′,N′-Tetramethylacridine-3,6-diamine), cyanine dimer dyes, cyanine monomer dyes, DAPI (4′,6-diamidino-2-phenylindole), DRAQ dyes, ethidium compounds, ethidium bromide, GelRed™, GelGreen™, Hoechst dyes, iodine compounds, 7-aminoactinomycin D, oxazole dyes, PicoGreen, propidium iodide (2, 7-Diamino-9-phenyl-10 (diethylaminopropyl)-phenanthridium iodide methiodide), SYBR dyes, SYTO dyes, TOTO™ dyes, thiozole dyes, thiazole orange homodimer (TOTO™-1), thiozole red (TO-PRO®-3), thiazole red homodimer (TOTO®-3), oxazole yellow (YO-PRO®-1), oxazole yellow homodimer (YOYO™-1), oxazole red (YO-PRO®-3), oxazole red homodimer (YOYO™-3), oxazole blue (PO-PRO™-1), oxazole blue homodimer (POPO™-1), TO Iodide (TO-PRO™-1), BOBO, JOJO, LOLO, At BO-PRO, JO-PRO, LO-PRO, or any combination of two or more thereof.

Methods of characterizing a population of particles according to aspects of the present disclosure include flowing the labeled extracted recombinant nucleic acids through a polymeric separation medium in a microchannel of a microfluidic device.

A microfluidic device used according to aspects of methods of the present disclosure allows for separation of labeled extracted recombinant nucleic acids and/or extracted labeled particle proteins and detection of the separated labeled extracted recombinant nucleic acids and/or extracted labeled particle proteins.

According to aspects of the present disclosure, the microfluidic device is a capillary electrophoresis system allows for separation of labeled extracted recombinant nucleic acids and/or extracted labeled particle proteins and detection of the separated labeled extracted recombinant nucleic acids and/or extracted labeled particle proteins. According to aspects of the present disclosure, the microfluidic device is, or includes, a microfluidic chip.

A microfluidic device used according to aspects of methods of the present disclosure includes at least one microchannel and may include one or more receptacles in fluid communication with one or more microchannels. The microchannels and/or receptacles are included in the microfluidic device, for example, by etching, bonding, soft lithography, or molding into a material which is substantially insert with respect to the sieving matrix, dyes, nucleic acid and/or proteins, ceramics and semiconductors, such as glass or silicon, or a polymer, such as polydimethylsiloxane (PDMS). Some or all of the microchannels and/or receptacles may be connected in a network as desired and the microchannels and/or receptacles may be in fluid communication with one or more inputs and/or outputs to allow for input and/or output to/from the microfluidic device.

A microchannel of a microfluidic device is made of any material suitable for containing a separation medium and aliquot of sample while remaining inert to the separation medium and aliquot of sample, such as, but not limited to, glass, silicon, plastic, quartz, or mixtures of any two or more thereof.

In some aspects, the microchannel has a length in the range of about 25 millimeters to about 250 millimeters, such as 25 millimeters to about 35 millimeters, 35 millimeters to about 45 millimeters, 45 millimeters to about 55 millimeters, 55 millimeters to about 65 millimeters, millimeters to about 75 millimeters, 75 millimeters to about 85 millimeters, 85 millimeters to about 95 millimeters, 95 millimeters to about 100 millimeters, 100 millimeters to about 110 millimeters, 110 millimeters to about 120 millimeters, 120 millimeters to about 130 millimeters, 130 millimeters to about 140 millimeters, 140 millimeters to about 150 millimeters, 150 millimeters to about 160 millimeters, 160 millimeters to about 170 millimeters, 170 millimeters to about 180 millimeters, 180 millimeters to about 190 millimeters, 190 millimeters to about 200 millimeters, 200 millimeters to about 210 millimeters, 210 millimeters to about 220 millimeters, 220 millimeters to about 230 millimeters, 230 millimeters to about 240 millimeters, or 240 millimeters to about 250 millimeters.

In some aspects, the microchannel has an internal diameter of between about 1 micron to about 10 millimeters, such as about 1 micron to about 10 microns, about 10 microns to about 20 microns, about 20 microns to about 30 microns, about 30 microns to about 40 microns, about 40 microns to about 50 microns, about 50 microns to about 60 microns, about 60 microns to about 70 microns, about 70 microns to about 80 microns, about 80 microns to about 90 microns, about 90 microns to about 100 microns, about 100 microns to about 200 microns, about 200 microns to about 300 microns, about 300 microns to about 400 microns, about 400 microns to about 500 microns, about 500 microns to about 600 microns, about 600 microns to about 700 microns, about 700 microns to about 800 microns, about 800 microns to about 900 microns, about 900 microns to about 1 millimeter, or about 1 millimeter to about 10 millimeters.

According to aspects of the present disclosure, the polymeric separation medium, also known as a sieving matrix, is, or includes, a polymer such as, but not limited to, one or more polyacrylamides, polyvinylpyrrolidinone, and agarose, or a mixture of any two or more thereof. Polyacrylamides that can be included in the sieving matrix include, but are not limited to, linear polyacrylamide, polydimethylacrylamide, polydiethylacrylamide, hydroxyethylcellulose, or a mixture of any two or more thereof. According to aspects of the present disclosure, the polymeric separation medium is, or includes, a polymeric gel such as, but not limited to, an acrylamide gel and an agarose gel. A polymer included in the sieving matrix is optionally cross-linked. Optionally, a denaturing agent is included in the sieving matrix. A denaturing agent included in the sieving matrix. A denaturing agent included in the sieving matrix can be a chaotropic agent, a detergent, or a mixture thereof

According to aspects of the present disclosure an included sieving matrix is configured to separate labeled viral nucleic acids from other materials which may be present in the aliquot of the sample and to obtain a signal from the labeled viral nucleic acids representative of the amount of labeled viral nucleic acids present in the aliquot of the sample. According to aspects of the present disclosure an included sieving matrix is configured to separate labeled viral proteins from other materials which may be present in the aliquot of the sample and to obtain a signal from the labeled viral proteins representative of the amount of labeled viral proteins present in the aliquot of the sample. Configuring the sieving matrix to separate different types of viral particles may include, but is not limited to, selection of one or more of: pore size, polymer type, polymer concentration, cross-linker, and extent of cross-linkage.

According to aspects of the present disclosure an included sieving matrix is configured to separate one or more of: labeled extracted AAV viral protein VP1, VP2 and VP3 from other materials which may be present in the aliquot of the sample and to obtain a signal from one or more of: labeled extracted AAV viral protein VP1, VP2 and VP3 representative of the amount of one or more of: labeled extracted AAV viral protein VP1, VP2 and VP3 present in the aliquot of the sample.

According to aspects of the present disclosure an included sieving matrix is configured to separate labeled extracted AAV viral protein VP3 from other materials which may be present in the aliquot of the sample and to obtain a signal from the labeled extracted AAV viral protein VP3 representative of the amount of labeled extracted AAV viral protein VP3 present in the aliquot of the sample.

The sieving matrix may be uniform with respect to pore size along the length of a microchannel in which it is disposed. Alternatively, the sieving matrix may be disposed in a non-uniform manner in the microchannel, such as in a smooth gradient of pore size; or in two or more blocks of uniform pore size to achieve a “step” gradient.

The concentration of the sieving polymer matrix used and its viscosity is adjusted according to the size of the nucleic acids and/or proteins to be detected. According to aspects of the present disclosure, and included sieving polymer matrix is used at a concentration of about 1% -10% wt/vol and the viscosity ranges between about 5-100 cSt. According to aspects of the present disclosure for determining the ratio of empty:full, empty: partially full, or full: partially full AAV particles, a concentration of about 2% to about 6% wt/vol sieving polymer matrix and a viscosity of about 40-53 cSt is used.

Typically, flowing the aliquot through the polymeric separation medium in the microchannel is performed at a temperature in the range of about 4° C. to about 30° C. According to aspects of the present disclosure, flowing the aliquot through the polymeric separation medium in the microchannel is performed at a temperature in the range of about 4° C. to about 10° C. to about 15° C., 15° C. to about 20° C., 20° C. to about 25° C., 25° C. to about 30° C., or about to about 40° C.

Typically, flowing the aliquot through the polymeric separation medium in the microchannel includes flow into a detection region in fluid communication with the microchannel, the detection region in signal communication with a sensor capable of detecting a signal from the detectable label of the labeled extracted recombinant nucleic acids and/or a signal from the detectable label of the labeled extracted particle proteins.

The aliquot of the sample is flowed through the polymeric separation medium to separate labeled extracted recombinant viral nucleic acids or particle proteins. Flow through the polymeric separation medium from one position in the microchannel towards a distant position in the microchannel is achieved by capillary action, diffusion, hydrodynamic action, and/or promoted by application of pressure gradient, voltage gradient, or a combination of two or more thereof. Where the microchannel is approximately columnar flow through the polymeric separation medium from one end of the microchannel towards a distant opposed end of the microchannel is achieved by capillary action, diffusion, hydrodynamic action, and/or promoted by application of pressure gradient, voltage gradient, current gradient, or a combination of two or more thereof along the length of the microchannel. According to aspects of the present disclosure, a voltage gradient or current gradient is applied to promote flow through the polymeric separation medium from one end of the microchannel towards a distant opposed end of the microchannel.

The aliquot of the sample is flowed through the polymeric separation medium in the microchannel into a detection region in fluid communication with the microchannel, the detection region in signal communication with a sensor capable of detecting a signal from the detectable label of the labeled extracted recombinant nucleic acids and/or labeled extracted particle protein.

Non-limiting examples of sensors capable of detecting a signal from the detectable label of the labeled recombinant nucleic acid and/or labeled extracted particle proteins include a charge-coupled device (CCD), electron-multiplying CCD, photomultiplier tube, photosensitive diode, a complementary metal-oxide semiconductor (CMOS), an intensified charge-coupled device (ICCD), and an avalanche photodiode.

According to aspects of the present disclosure, the detected signal could be an absorption or a fluorescent signal emitted from a label as a result of contact with electromagnetic radiation which excites the label.

The detected signal may be measured to determine one or more quantitative aspects of the sample, such as one or more of: a) an amount of time taken by the labeled extracted recombinant nucleic acids to flow through the polymeric separation medium in the microchannel into the detection region, indicative of size of the recombinant nucleic acids in the fluid sample, and/or b) strength of the signal of the detectable label in the detection region, representative of the amount of labeled extracted recombinant nucleic acids present, and indicative of concentration of the labeled extracted recombinant nucleic acids in the fluid sample.

According to aspects of the present disclosure, the detector is operably connected to a computer which stores and manipulates the detected signal information, for example, to calculated one or more parameters relevant to the particles in the fluid sample. According to aspects of the present disclosure, the detector is operably connected to a computer which stores and manipulates the detected signal information, for example, to compare the detected signal information a) to a reference standard representative of full particles and, based on the comparison, determining a ratio of full particles to partially full particles, producing a first assay result and/or comparing b) to a reference standard representative of full particles and, based on the comparison, determining a ratio of full particles to empty particles in the fluid sample.

According to aspects of the present disclosure, the detector is operably connected to a computer which stores and manipulates the detected signal information, for example, to compare the detected signal information relating to particle nucleic acids a) to a reference standard representative of full particles and, based on the comparison, determining a ratio of full particles to partially full particles, producing a first assay result and/or comparing b) to a reference standard representative of full particles.

According to aspects of the present disclosure, the detector is operably connected to a computer which stores and manipulates the detected signal information, for example, to compare the detected signal information relating to particle proteins representing: c) an amount of time taken by the labeled extracted proteins to flow through the polymeric separation medium in the microchannel into the detection region, and d) strength of the signal of the detectable label representative of the amount of labeled extracted proteins present; comparing c) and d) to a reference standard, the reference standard representing a known amount of the protein, thereby determining an amount of protein in the sample, and thereby producing a second assay result; and comparing the first assay result and the second assay result, thereby determining one or more of: 1) a ratio of full viral particles to empty viral particles in the fluid sample, 2) a ratio of full viral particles or empty viral particles to partially full viral particles, and 3) a ratio of viral particles containing an intact recombinant genome to viral particles containing an incomplete recombinant genome, thereby characterizing the population of recombinant viral particles in the fluid sample.

Methods of characterizing a population of particles putatively containing recombinant nucleic acids in a fluid sample are provided according to aspects of the present disclosure. The term “fluid” as used herein in reference to a sample refers to a liquid, gel, or combination of liquid and gel, that can be flowed through a microchannel. According to aspects of the present disclosure, the fluid is an aqueous fluid. According to aspects of the present disclosure, the fluid is an aqueous buffer such as, but not limited to, a Tris buffer, a Tricine buffer, a citrate buffer, a HEPES buffer, a carbonate buffer, a phosphate buffer, a MOPS buffer, a TAPS buffer, and an acetate buffer. Typically, the pH of the aqueous buffer is in the range of about pH 5.0 to about pH 9. According to aspects of the present disclosure, the pH of the aqueous buffer is about pH 5.0 to about pH 5.5, pH 6.0 to about pH 6.5, about pH 6.5 to about pH 7.0, about pH 7.0 to about pH 7.5, about pH 7.5 to about pH 8.0, about pH 8.0 to about pH 8.3, or about pH 8.3 to about pH 9.

The term “sample” as used herein refers to any material that includes, or may include, particles of interest.

Optionally, a sample is purified prior to introducing the aliquot of the fluid sample into a microchannel of a microfluidic device. The term “purified” as used herein refers to reduction of at least some contaminating substances such as materials associated with production of the particles in the sample, including but not limited to, helper virus, helper virus proteins, host cell proteins, and host cell debris. According to aspects of the present disclosure, a sample is purified such that contaminating substances in the sample are reduced by at least 10% or more, at least 20% or more, at least 30% or more, at least 40% or more, at least 50% or more, at least 60% or more, at least 70% or more, at least 80% or more, at least 90% or more, or at least 95% or more.

The term “aliquot of the fluid sample” refers to a portion of the fluid sample for analysis. The aliquot typically has a volume in the range of about 0.5 microliter to about 50 microliters. According to aspects of the present disclosure, the aliquot has a volume in the range of about 0.5 microliter to about 5 microliters, about 1 microliter to about 10 microliters, about 1 microliter to about 20 microliters, about 5 microliters to about 10 microliters, about 5 microliters to about 15 microliters, about 5 microliters to about 20 microliters, about 10 microliters to about microliters, about 15 microliters to about 20 microliters, about 25 microliters to about 30 microliters, about 30 microliters to about 35 microliters, about 35 microliters to about 40 microliters, about 40 microliters to about 45 microliters, or about 45 microliters to about 50 microliters. According to aspects of the present disclosure, the aliquot has a volume in the range of about 0.5 microliter, about 1 microliter, about 2 microliters, about 3 microliters, about 4 microliters, about 5 microliters, about 6 microliters, about 7 microliters, about 8 microliters, about 9 microliters, or about 10 microliters.

According to aspects of the present disclosure, viral particles in the fluid sample are present in an amount of about 1×10¹⁰ viral viral particles/milliliter to about 1×10¹⁴ viral particles/milliliter. According to aspects of the present disclosure, viral particles in the fluid sample are present in an amount of about 1×10¹⁰ viral particles/milliliter to about 0.5×10¹¹ viral particles/milliliter, about 0.5×10¹¹ viral particles/milliliter to about 1×10¹¹ viral particles/milliliter, 1×10¹¹¹ viral particles/milliliter to about 0.5×10¹² viral particles/milliliter, about 0.5×10¹² viral particles/milliliter to about 1×10¹² viral particles/milliliter, about 1×10¹² viral particles/milliliter to about 0.5×10¹³ viral particles/milliliter, about 0.5×10¹³ viral particles/milliliter to about 1×10¹³ viral particles/milliliter, about 1×10¹³ viral particles/milliliter to about 0.5×10¹⁴ viral particles/milliliter, or about 0.5×10¹⁴ viral particles/milliliter to about 1×10¹⁴ viral particles/milliliter.

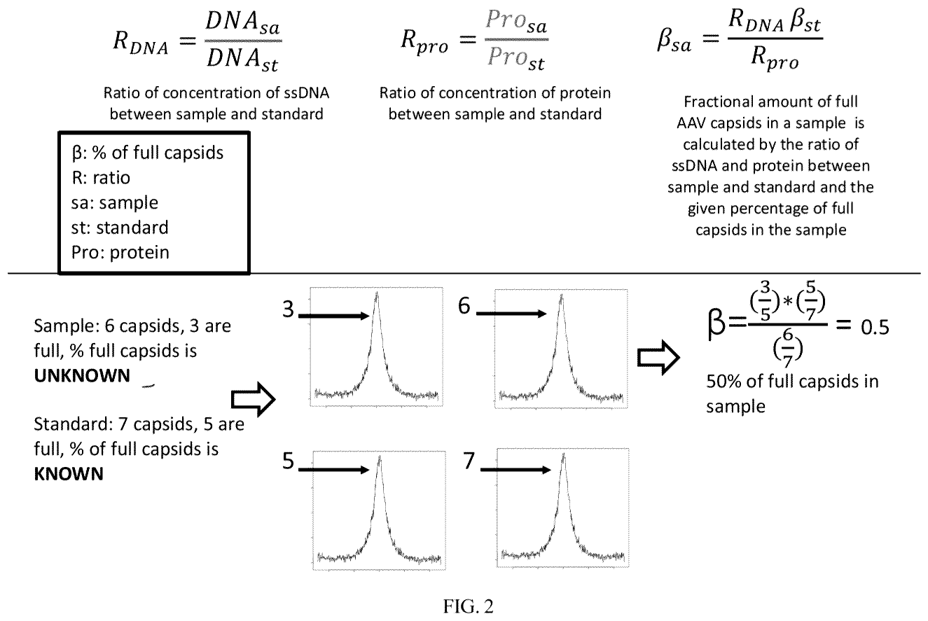

Methods of characterizing a population of viral particles in a sample are provided according to aspects of the present disclosure which include microfluidic electrophoretic assay of viral nucleic acids in the sample. The assay produces an assay result representing the amount of viral nucleic acids in the sample; and the assay result is compared to a reference standard, the reference standard representing a known amount of viral nucleic acids. The amount of nucleic acid may be expressed as concentration of nucleic acid in the sample and the reference standard. Comparison of the assay result to the reference standard provides a ratio of sample viral nucleic acids to reference standard, see FIG. 2.

According to aspects of the present disclosure, a ratio of the amount of viral protein in the sample to the amount of protein in a reference standard is generated. The amount of protein may be expressed as concentration of protein in the sample and the reference standard. Comparison of the amount of viral protein in the sample to the reference standard provides a ratio of sample viral protein to reference standard protein, see FIG. 2.

The amount of protein in the sample and/or standard is calculated using information about the number of viral particles in the sample and/or reference standard without assaying protein in the sample and/or reference standard according to aspects of the present disclosure.

Optionally, the amount of protein in the sample and/or standard is assayed by microfluidic electrophoretic assay according to aspects of the present disclosure.

A ratio, and/or fractional amount, of full viral capsids in the sample is determined by calculating the ratio of viral nucleic acids and viral protein in the sample compared to nucleic acids and protein in the reference standard according to aspects of the present disclosure.

According to particular aspects of the present disclosure, methods of characterizing a population of viral particles in a sample to determine a ratio, or fractional percentage, of full viral capsids, partially full viral capsids, and/or empty viral capsids in the sample are provided which include extracting viral nucleic acids from the sample; and assaying the extracted viral nucleic acids by microfluidic electrophoretic assay.

According to particular aspects of the present disclosure, methods of characterizing a population of viral particles in a sample to determine a ratio, or fractional percentage, of full viral capsids to empty viral capsids in the sample are provided which include extracting viral nucleic acids from the sample; assaying the extracted viral nucleic acids by microfluidic electrophoretic assay; extracting viral proteins from the sample; assaying the extracted viral proteins by microfluidic electrophoretic assay; calculating a ratio of nucleic acid concentration in the sample to nucleic concentration in the reference standard, calculating a ratio of protein concentration in the sample to protein concentration in the reference standard, and calculating a fractional amount of full capsids in the sample according to:

$$β = \frac{N ( f )}{N ( f ) + N ( e )} = \frac{{β}_{s} {R}_{DNA}}{{R}_{p}} , \qquad ( 6 )$$

wherein N is a total number of capsids, f and e refer to full and empty, respectively, βs is the percent of full capsids in the reference standard, R_{DNA} is the ratio of concentration of viral nucleic acids in the sample to the concentration of viral nucleic acids in the reference standard, and R_{P} is the ratio of concentration of viral proteins in the sample to the concentration of viral proteins in the reference standard.

According to aspects of the present disclosure, the sample contains AAV capsids and is assayed to determine the ratio of full AAV capsids to empty AAV capsids or vice versa, full AAV capsids to partially full AAV capsids or vice versa, partially full AAV capsids to empty AAV capsids or vice versa, or any combination thereof.

Kits for characterizing nucleic acid content of a population of particles putatively containing recombinant nucleic acids in a fluid sample are provided according to aspects of the present disclosure, which include: a nucleic acid label, a gel, a nucleic acid ladder standard, a nucleic acid storage buffer, a nucleic acid sample buffer, a proteinase, and a denaturing agent. According to aspects of the present disclosure, the nucleic acid label is a fluorescent nucleic acid intercalator. According to aspects of the present disclosure, the proteinase is a serine protease.

Kits for characterizing nucleic acid content of a population of particles putatively containing recombinant nucleic acids in a fluid sample are provided according to aspects of the present disclosure, which include: a nucleic acid label, a gel, a nucleic acid ladder standard, a nucleic acid storage buffer, a nucleic acid sample buffer, a proteinase, a denaturing agent, a protein dye, a protein standard, a protein storage buffer, a protein sample buffer, and a wash buffer. According to aspects of the present disclosure, the nucleic acid label is a fluorescent nucleic acid intercalator. According to aspects of the present disclosure, the proteinase is a serine protease.

Kits for characterizing nucleic acid content of a population of particles putatively containing recombinant nucleic acids in a fluid sample are provided according to aspects of the present disclosure, wherein the proteinase is selected from the group consisting of: proteinase K, subtilisin, trypsin, chymotrypsin, thrombin, plasmin, elastase, and lactoferrin.

Kits for characterizing nucleic acid content of a population of particles putatively containing recombinant nucleic acids in a fluid sample are provided according to aspects of the present disclosure, wherein the denaturing agent is a chaotropic agent, a detergent, or a mixture thereof.

Kits for characterizing nucleic acid content of a population of particles putatively containing recombinant nucleic acids in a fluid sample are provided according to aspects of the present disclosure contain at least one proteinase and at least one chaotropic agent, wherein the at least one proteinase is proteinase K and the at least one chaotropic agent is urea.

Kits for characterizing nucleic acid content of a population of particles putatively containing recombinant nucleic acids in a fluid sample are provided according to aspects of the present disclosure include one or more standards. According to aspects of the present disclosure, an included standard may be a sample including a known ratio of full particles:empty particles, a known ratio of full particles:partially full particles, or a known ratio of empty particles:partially full particles, two or more thereof, or all three thereof. According to aspects of the present disclosure, an included standard may be a sample including a known ratio of full AAV particles:empty AAV particles, a known ratio of full AAV particles:partially full AAV particles, or a known ratio of empty AAV particles:partially full AAV particles, two or more thereof, or all three thereof.

Embodiments of inventive compositions and methods are illustrated in the following examples. These examples are provided for illustrative purposes and are not considered limitations on the scope of inventive compositions and methods.

### EXAMPLES

### Example 1

### Samples and Sample Preparation

AAV8 full and empty reference standards were purchased from Vigene Biosciences (Vigene Biosciences, Rockville, MD). The full reference standard had a titer of 7.97×10¹¹ genome copies (GC)/mL at a full fraction of 75%, which is the equivalent to 1.07×10¹² viral particles (VP)/mL. The latter was estimated by accounting for the empty particles, which would not contribute to the genome copies. On the other hand, the empty reference standard was purchased with a titer of 1.44×10¹² VP/mL at an empty fraction of 96%. Since this value includes both full and empty particles, the titer of only empty particles in this sample was estimated to be 1.38×10¹² VP/mL. In the case of both the full and the empty reference standards, per the certificate of analysis issued by the provider (Vigene Biosciences), the quality control was conducted via SYBR Green qPCR and ELISA, combined with TEM to determine the percentage of full capsids in the sample. While the vendor did not provide a standard deviation for their estimations, a 10% deviation will be assumed, when needed, for statistical analysis, which may be consistent with the analytical tools used for their capsid ratio estimation. For the experiments comparing full to empty samples, the reference samples were used. For experiments that used samples having varying percentages of full viral particles, these samples were prepared accounting for the empty particles in the full reference standard, and for the full particles in the empty reference standard. Furthermore, samples were stored in single-use aliquots at −80° C. to ensure sample stability and degradation were not affecting the outcomes of the experiments.

### Methods

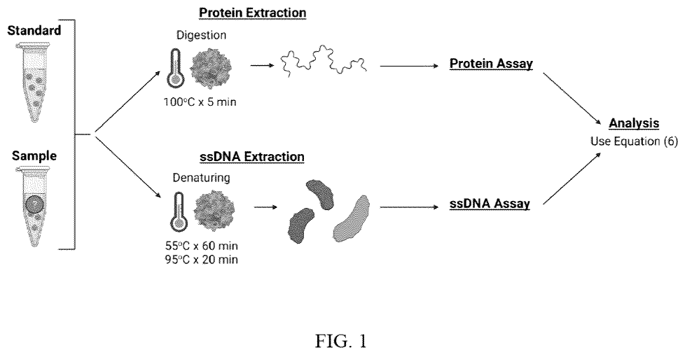

In this example, the GX Touch II LabChip system (PerkinElmer, Waltham, MA), a microfluidic electrophoretic separation system, was used to characterize samples containing virus particles to determine a ratio of full:empty viral particles in the samples. For the capsid protein experiments, the standard protocol of the LabChip ProteinExpress assay in the the GX Touch II LabChip system was used, shown schematically in FIG. 1, top. Specifically, the high sensitivity protocol was followed, which requires 5 μL of sample, using the optional reducing buffer and incubation at 100° C. for 5 minutes to extract protein. Each sample was analyzed three times, and each time 20 nL were transferred from the well plate onto the detection chip. For the ssDNA analysis, a custom ssDNA chip assay was used. First, the AAV samples were digested 1:1 (5 μL of AAV and 5 μL of the digestion mixture) with a proteinase K digestion mixture (10 μL of proteinase K were diluted with 90 μL of 2M Urea) for 60 minutes at 50° C., followed by a proteinase K deactivation for 20 min at 95° C. (note that the samples were not diluted 1:10 in sample buffer), shown schematically in FIG. 1, bottom. While this example focuses on the detection of ssDNA, AAV could also contain self-complementary DNA, which can also be detected with minor assay modifications. The samples were then analyzed with a customized ssDNA assay script to amplify the signal and allow for the detection of samples with lower genomic content. Each sample was analyzed three times, and each time 20 nL were transferred from the well plate onto the detection chip. While 5 μL of sample were used for each assay, if needed, this volume can be reduced significantly without interfering with the assay. The statistical analysis for this study was conducted using GraphPad Prism 9, and the figures were made using GraphPad and/or BioRender.

Quantitation of both proteins and ssDNA of the viral particles in the samples was performed according to aspects of a method of the present disclosure in this example, although analysis of protein content is optional since protein content of both full and empty capsids is equivalent.

Methods for the determination of the percentage of full capsids in a sample containing viral particles according to aspects of the present disclosure are described.

According to aspects of the present disclosure only a few microliters, such as 3-10 uL, of a standard (S) with a known fraction of full capsids is used.

Mathematical formulas to relate protein and DNA concentrations in samples and viral particle standards are used according to aspects of a method of the present disclosure, described hereinbelow. Mathematical Formulation to Estimate the Percentage of Full Particles

In this example, samples containing empty, full, and/or partially full AAV capsids were used. The empty, full, and partial AAV capsids have icosahedral symmetry. AAV particles are assembled from viral proteins (VP1, VP2, VP3) and genomic material (ssDNA).

The total number of AAV capsids in the sample and standard are denoted by N and Nₛ, respectively. Hence, the number of full and empty capsids can be described using the following equations (1) and (2), where f stands for full, and e stands for empty:

N=N(f)+N(e) (1)

Nₛ=Nₛ(f)+Nₛ(e) (2)

Since the protein subunits (VP1, VP2, VP3) of the AAV capsids are the same for both full and empty AAV, it is safe to assume that each capsid is composed of α ug of total proteins. Hence, the ratio of the concentration, Rₚ, of proteins in the sample (c(protein) and standard (cₛ(protein)) can be expressed in equation (3) as:

$${R}_{p} = \frac{c ( protein )}{{c}_{s} ( protein )} = \frac{α N}{α {N}_{s}} = \frac{N}{{N}_{s}} \qquad ( 3 )$$

Similarly, a single ssDNA insert per full AAV capsid is assumed to obtain the following ratio, R_{DNA}, for the concentration of ssDNA, in the sample (c(ssDNA)) and standard (cₛ(SSDNA)):

$${R}_{DNA} = \frac{c ( ss DNA )}{{c}_{s} ( ss DNA )} = \frac{N ( f )}{{N}_{s} ( f )} \qquad ( 4 )$$

Since the standard comes with a known percent of full capsids, βs, which can be defined as:

$${β}_{s} = \frac{{N}_{s} ( f )}{{N}_{s} ( f ) + {N}_{s} ( e )} \qquad ( 5 )$$

the fraction of full capsids, β, is obtained using the following relation:

$$β = \frac{N ( f )}{N ( f ) + N ( e )} = \frac{{β}_{s} {R}_{DNA}}{{R}_{p}} \qquad ( 6 )$$

Hence, the percentage full AAV estimate is obtained according to aspects of the present disclosure by measurements of relative concentrations of total protein and ssDNA using microfluidics electrophoresis with samples and one or more standards. This method is independent of the total capsid concentrations in samples or standards. This is often the most significant limitation in other techniques. If there is any error in the estimation obtained using Equation (6), it only arises from the concentration ratio accuracy errors in the electrophoresis method, not from the resolution accuracy of the method. In other words, rather than obtaining the total protein concentration of a sample from the protein assay (full and empty) and subtracting the concentration obtained from the DNA assay (only full), which would have a compounded error rate from the use of two different assays, methods according to aspects of the present disclosure use a sample of known concentration to normalize both assays. Another key advantage of the ratio measurements is that as long as all samples within the assay are treated in the same way (diluted by the same factor, or heated for the same time), methods according to aspects of the present disclosure are independent of the concentration of the sample since the protein area will account for a difference in concentration.

### Capsid Protein Profile Characterization

For validation of methods of estimation of the percentage of full capsids in a sample according to the present disclosure, assessment of the protein and DNA profiles of AAV8 full (in a “full sample” containing 75% full, 25% empty) and empty (in an “empty sample” containing 96% empty, 4% full) reference standards was performed. It was confirmed by experimental analysis that the amount of protein in full and empty capsid reference standards was preserved for a given concentration. To do this, the AAV standards were denatured using a reducing buffer containing DTT. The concentration of the empty reference standard was adjusted to match the concentration of the full reference standard (1.07×10¹² VP/mL) by diluting it with 1× PBS. The dilution was conducted keeping both the viral concentration and the salt composition, which can affect labeling efficiency and sample conductivity, constant.

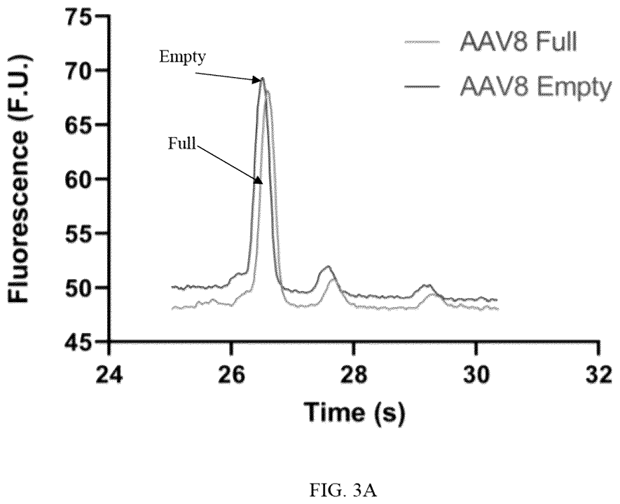

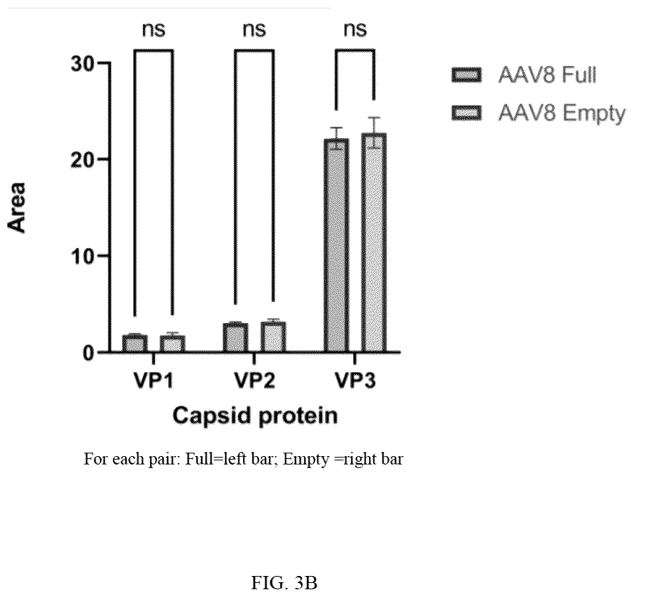

Based on the electropherogram shown in FIG. 3A, and as seen in the summarized corresponding areas under the curve for VP1, VP2 and VP3 (FIG. 3B), there was no statistical difference between the VP peaks of the full and empty samples. Moreover, based on literature and normalizing it to their respective molecular weights, VP1 is expected to represent 11.3% of the total capsid area, VP2 9.5%, and VP3 79.2%. In the analysis of this example, the full sample VP1 represents 6.7%, 11.3% for VP2, and 82.1% for VP3. Similarly, the empty sample VP1 represents 6.3%, VP2 11.6%, and VP3 82.1%. The presence of these VP ratios that differ from the expected 1:1:10 ratio highlights the stochastic nature of the VP capsid protein ratios. Therefore, to mitigate the impact of the varying VP ratios across samples and even within a given batch, the area under the three VP peaks was integrated to have a method that is independent of the VP ratios. According to aspects of methods of the present disclosure, one, two, or all three of these peaks (i.e. VP1 peak, VP2 peak, and VP3 peak) can be used for the estimation. Alternatively, the VP3 peak alone can be used since molecules with faster electrophoretic mobilities usually yield higher quantification accuracy as there is less time for the molecules to diffuse (broader peak), and higher concentrations, which increases peak area and height, can help visualize the tails of the peak caused by diffusion.

Moreover, if the sample and standard (usually provided) concentrations are known, these values can be inputted as the concentration in a modified Equation (3) instead of the protein area obtained in the Protein Express assay detailed above (refer to Equations 7-8). However, if the concentration is unknown, this approach can be used not only to estimate the percentage of full capsids in the sample, but also to estimate the total concentration of the sample, as highlighted in Tables 1A and 1B. Using the area under the curve of the VP protein peaks of the standard and of the sample, as well as the concentration of the standard, the total sample concentration was estimated with an error rate of 1-16%, and an average error rate of 6%. The present results indicate that the L.O.D. in this example lies between 5×10¹¹ and 1×10¹² VP/mL. The current L.O.D. for the ssDNA assay in this example has been estimated to be >1×10¹¹ GC/mL, which will generally place the total protein content within the desired range.

Table 1A. Estimation of sample total protein concentration based on the VP peaks area under the curve of the standard (known concentration) and the area under the curve of the sample. The predicted total concentration was estimated using Equation (3). Each set refers to an independent run in which samples were analyzed in triplicate.

TABLE 1A

| | | | | |
|---|---|---|---|---|
| | Total | | Predicted Total | |
| Sample | Concentration | Protein | Concentration | Prediction |
| # | (VP/mL) | Area | (VP/mL) | Error Rate |

Set 1

| | | | | |
|---|---|---|---|---|
| 1-0 | 1.07 × 10¹² | 41.71 ± 1.52 | Standard | Standard |
| 1-1 | 1.07 × 10¹² | 42.30 ± 3.04 | 1.08 × 10¹² | 1% |
| 1-2 | 1.07 × 10¹² | 43.67 ± 3.40 | 1.12 × 10¹² | 5% |

Set 2

| | | | | |
|---|---|---|---|---|
| 2-0 | 1.07 × 10¹² | 39.60 ± 3.01 | Standard | Standard |
| 2-1 | 1.20 × 10¹² | 41.16 ± 2.37 | 1.11 × 10¹² | 7% |
| 2-2 | 1.33 × 10¹² | 42.90 ± 2.68 | 1.12 × 10¹² | 16% |

Set 3

| | | | | |
|---|---|---|---|---|
| 3-0 | 1.07 × 10¹² | 31.32 ± 1.68 | Standard | Standard |
| 3-1 | 1.07 × 10¹² | 31.86 ± 1.08 | 1.08 × 10¹² | 2% |
| 3-2 | 1.07 × 10¹² | 33.52 ± 2.77 | 1.14 × 10¹² | 7% |

Note that, as long as the sample and standard (usually provided) concentrations are known, these values can be input as the concentration in a modified Equation (3) instead of the VP3 area peak obtained in the Protein Express assay detailed herein (refer to Equations 7-8). However, if the concentration is unknown, this approach can be used not only to estimate the percentage of full capsids in the sample, but also to estimate the total concentration of the sample, as highlighted in Table 1B. Using the area under the curve of the VP3 peak of the standard and of the sample, as well as the concentration of the standard, the total sample concentration was estimated according to aspects of methods of the present disclosure with an error rate of 4-19%, and an average error rate of 11%.

Table 1B. Estimation of sample total protein concentration based on the VP3 area under the curve of the standard (known concentration) and the area under the curve of the sample. The predicted total concentration was estimated using Equation (3). Note that each set refers to an independent run in which samples were analyzed in triplicate.

TABLE 1B

| | | | |
|---|---|---|---|
| Total | | Predicted Total | |
| Concentration | | Concentration | Prediction Error |
| (VP/mL) | Protein Area | (VP/mL) | Rate |

Set 1

| | | | |
|---|---|---|---|
| 5.33 × 10¹¹ | 6.74 ± 0.16 | Standard | Standard |
| 5.83 × 10¹¹ | 6.68 ± 0.15 | 5.28 × 10¹¹ | 10% |
| 6.43 × 10¹¹ | 7.01 ± 0.39 | 5.54 × 10¹¹ | 14% |

Set 2

| | | | |
|---|---|---|---|
| 1.07 × 10¹² | 26.58 ± 1.40 | Standard | Standard |
| 1.07 × 10¹² | 28.58 ± 2.05 | 1.15 × 10¹² | 8% |
| 1.07 × 10¹² | 30.61 ± 3.40 | 1.23 × 10¹² | 15% |

Set 3

| | | | |
|---|---|---|---|
| 1.07 × 10¹² | 23.76 ± 2.33 | Standard | Standard |
| 1.20 × 10¹² | 23.97 ± 2.63 | 1.07 × 10¹² | 10% |
| 1.33 × 10¹² | 24.01 ± 1.67 | 1.08 × 10¹² | 19% |

Set 4

| | | | |
|---|---|---|---|
| 1.07 × 10¹² | 22.16 ± 1.12 | Standard | Standard |
| 1.07 × 10¹² | 22.94 ± 1.15 | 1.10 × 10¹² | 4% |
| 1.07 × 10¹² | 24.93 ± 2.36 | 1.20 × 10¹² | 13% |

ssDNA Profile Characterization

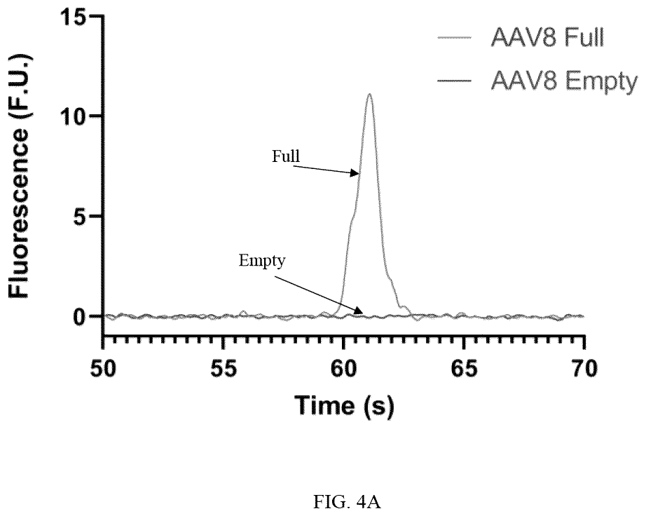

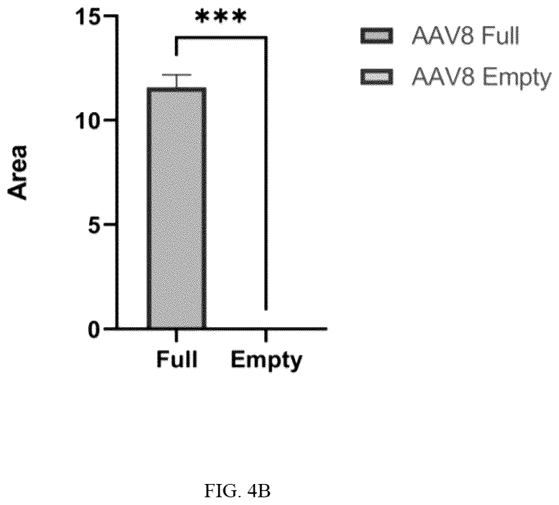

After confirming that the amount of capsid protein is preserved between full and empty capsids, as described above, the ssDNA content of the capsids was assessed. In this example, the sample viral particle concentration was normalized to 1.07×10¹² VP/mL and then the sample was digested using a standard Proteinase K digestion protocol as described above. As shown in FIGS. 4A and 4B, the full reference standard (75% full) exhibits a peak at around 62 s, while the empty standard (96% empty) failed to produce a peak. While it is not surprising that the empty sample did not have a peak, it must be noted that the empty sample should still contain approximately 4.26×10¹⁰ full capsids, which implies that the L.O.D. is greater than that in this example. Additional experiments were conducted establishing the L.O.D. at approximately 1×10¹¹ GC/mL. This limit is partly due to the lack of compatible assays optimized for the fluorescent analysis of ssDNA, therefore the labeling efficiency of the system will be limited.

Like the protein estimations generated using equation (3) shown in Tables 1A and 1B, the DNA information collected with this assay can be input into Equation (4) to estimate the number of full particles (or genome copies, GC) in the sample, as shown in Table 2. Using this approach, the prediction error rate ranged from 2-20%, with an average error rate of 8%.

Table 2: Estimation of sample genomic concentration based on the ssDNA area under the curve of the standard (known concentration) and the area under the curve of the sample. The predicted genomic concentration was estimated using Equation (4). Each set refers to an independent run in which samples were analyzed in triplicate.

TABLE 2

| | | | | |
|---|---|---|---|---|
| | | | Predicted | |
| | Genomic | | Genomic | |
| Sample | Concentration | DNA | Concentration | Prediction |
| # | (GC/mL) | Area | (GC/mL) | Error Rate |

Set 1

| | | | | |
|---|---|---|---|---|
| 1-0 | 7.97 × 10¹¹ | 11.32 ± 0.33 | Standard | Standard |
| 1-1 | 5.33 × 10¹¹ | 8.02 ± 0.64 | 5.64 × 10¹¹ | 6% |
| 1-2 | 2.66 × 10¹¹ | 3.58 ± 0.52 | 2.52 × 10¹¹ | 5% |

Set 2

| | | | | |
|---|---|---|---|---|
| 2-0 | 7.97 × 10¹¹ | 10.76 ± 0.32 | Standard | Standard |
| 2-1 | 5.38 × 10¹¹ | 7.12 ± 0.70 | 5.27 × 10¹¹ | 2% |
| 2-2 | 2.77 × 10¹¹ | 3.64 ± 0.60 | 2.70 × 10¹¹ | 3% |

Set 3

| | | | | |
|---|---|---|---|---|
| 3-0 | 7.97 × 10¹¹ | 22.47 ± 2.57 | Standard | Standard |
| 3-1 | 5.33 × 10¹¹ | 13.55 ± 2.00 | 4.80 × 10¹¹ | 10% |
| 3-2 | 2.66 × 10¹¹ | 6.00 ± 1.61 | 2.13 × 10¹¹ | 20% |

Capsid Protein and ssDNA Profiles of Samples with Varying Full Percentages

For estimating the percentage of full capsids in an unknown AAV8 sample, information was collected from samples with varying percentages of full capsids, from 75% to 4%. In this example, both samples were normalized to a total protein concentration of 1.07×10¹² VP/mL, and the full reference standard was mixed with the empty standard at different ratios to achieve the desired percentages of full.

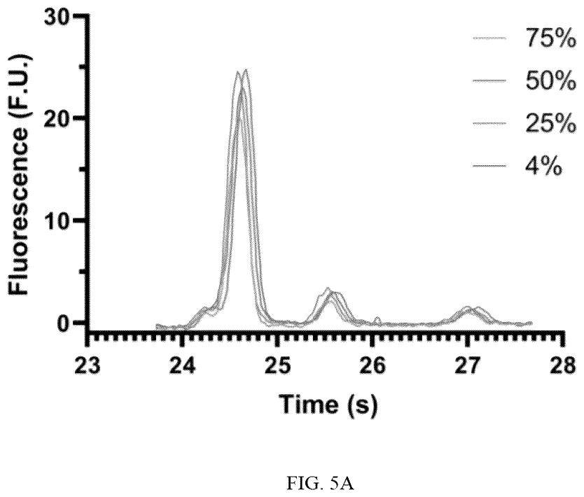

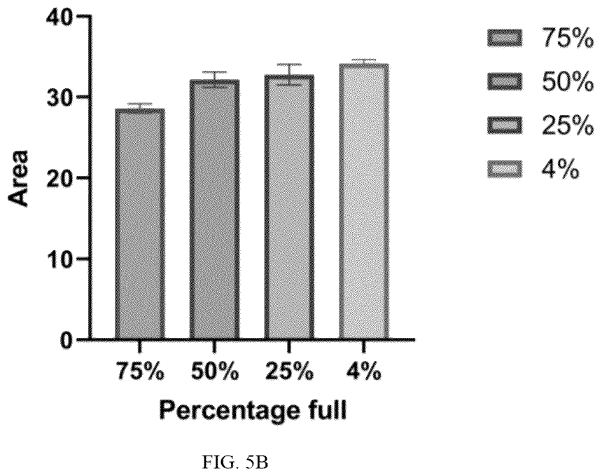

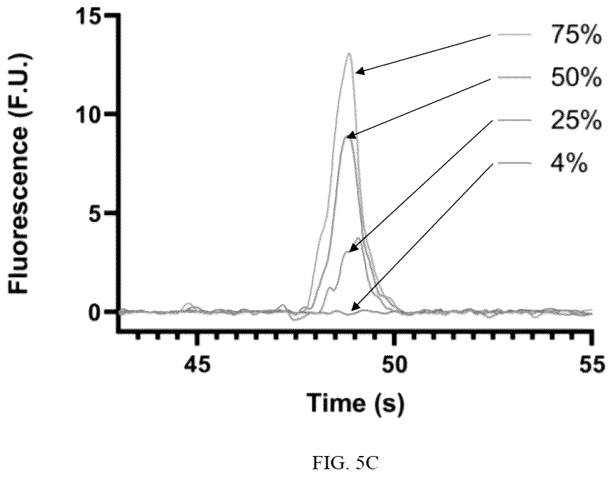

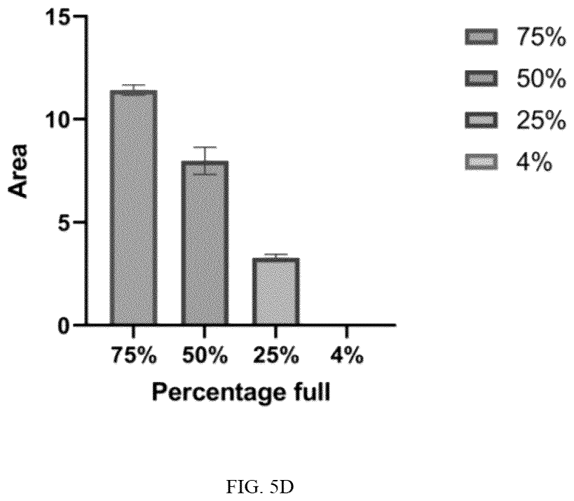

FIGS. 5A, 5B, 5C, and 5D show results of protein and nucleic acid analysis of samples with different percentages of full capsids, including 75%, 50%, 25%, and 4%. FIG. 5A: Electropherogram representative of VP3, VP2 and VP1 capsid proteins, from smallest to largest weight (left to right) of both full and empty samples. FIG. 5B: Summarized integrate area under the curve of the VP protein peaks for the four samples of varying full capsid percentages. FIG. 5C: Representative electropherogram of the genetic material of the four samples, where the DNA peak is observed to decrease as the percentage of full capsids decreases. FIG. 5D: Summarized area under the curve for the genetic material peak of each sample, note that for all three runs the empty AAV sample did not produce a peak.

As expected, while the protein concentration remains approximately the same with an average total VP area of 28.6 to 34.16 (FIGS. 5A and 5B), the ssDNA concentration decreases as the number of full capsids decreases, with average areas going from 11.4 to 0.0 (FIGS. 5C and 5D).

Experimental data was collected on three independent sets of protein and ssDNA assays, analyzed in triplicate. Using the protein and ssDNA information from the full reference standard (75% full) and from the samples, Equation (6) was used to estimate (3, or the predicted percentage of full capsids. The results for each set are shown in Table 3.

Table 3. Compiled protein and ssDNA data collected and analyzed from three different sets of experiments.

TABLE 3

| | | | | | |
|---|---|---|---|---|---|
| | Per- | | | Predicted | |
| Sample | centage | Protein | DNA | Percentage | Prediction |
| # | Full | Area | Area | Full | Error |

Set 1

| | | | | | |
|---|---|---|---|---|---|
| 1-0 | 75% | 41.71 ± 1.52 | 11.32 ± 0.33 | Standard | Standard |
| 1-1 | 50% | 42.30 ± 3.04 | 8.02 ± 0.64 | 52% | 2% |
| 1-2 | 25% | 43.67 ± 3.40 | 3.58 ± 0.52 | 23% | 2% |

Set 2

| | | | | | |
|---|---|---|---|---|---|
| 2-0 | 75% | 23.76 ± 2.33 | 10.76 ± 0.32 | Standard | Standard |
| 2-1 | 45% | 23.97 ± 2.63 | 7.12 ± 0.70 | 48% | 3% |
| 2-2 | 21% | 24.01 ± 1.67 | 3.64 ± 0.60 | 24% | 3% |

Set 3

| | | | | | |
|---|---|---|---|---|---|
| 3-0 | 75% | 22.16 ± 1.12 | 22.47 ± 2.57 | Standard | Standard |
| 3-1 | 50% | 22.94 ± 1.15 | 13.55 ± 2.00 | 44% | 6% |
| 3-2 | 25% | 24.93 ± 2.36 | 6.00 ± 1.61 | 19% | 6% |

Currently, the average prediction deviation of the model from the actual percentage is 4%, ranging from 2-6% with a standard deviation of 2%.

Alternatively, if the sample concentration is known within an acceptable margin of error, the error rate and sample analysis time of the system can be significantly decreased by omitting the protein assay according to aspects of methods of the present disclosure. Assuming an error rate of 10% for the provided concentrations and full percentages of the samples, and consequently of our predictions since they are based on these values and is the reported error rate of the detection platform, the following modified version of Equation (6) was used to estimate the percentage of full capsids using the ssDNA area and the provided concentrations:

$${R}_{concentration} = \frac{c}{{c}_{s}} \qquad ( 7 )$$

$$\\ \begin{matrix}β = \frac{N ( f )}{N ( f ) + N ( e )} = \frac{{β}_{s} {R}_{DNA}}{{R}_{concentration}} & ( 8 )\end{matrix}$$

where R_{concentration} is used to replace Rₚ since, as indicated by the data in Table 2, the sample concentration is related to the protein area.

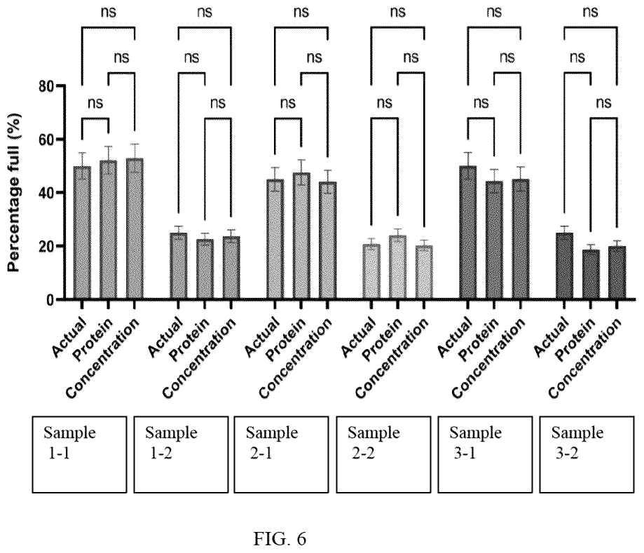

In order to assess the specificity of this alternative aspect of methods of the present disclosure, the actual (reported) percentage of full capsids of each sample was compared to the predictions obtained using Equation (6), referred to as “Protein Area Prediction”, and Equation (8), referred to as Concentration Prediction, as highlighted in FIG. 6. Note that while this aspect of methods of the present disclosure method bypasses the need for protein analysis, it is still highly dependent upon the ssDNA analysis, as indicated by Equation (8).

After analyzing the results obtained using both approaches, a decrease from 4% (protein area prediction method) to 3% in average prediction deviation was observed when using the concentration prediction method, ranging from 1-9%. In other words, when the total sample concentration is provided, both the error rate and turnaround time can be decreased. Importantly, the decrease in turnaround time will be limited since the longest incubation is needed for the extraction of ssDNA from the capsid. When the known concentrations where used, the standard deviation of the predictions increased slightly from 2% to 3%.

Thus, assessment of ssDNA in capsids and normalizing the findings to capsid protein (either by measurement of the protein or by non-measured calculated values of protein amount) provides desired information about the ratio of full to empty capsids, and therefore the percentage of full capsids, in samples.

Relationship between the protein and ssDNA profiles of full and empty standards was used in a method to estimate the percentage of full capsids in an AAV8 sample of unknown concentration and composition according to the present disclosure. First, primary assays were validated by comparing measured findings to literature values. While the protein assay did not yield a high accuracy in the ratios for VP1 and VP2, which is not surprising based on their lower concentration and electrophoretic mobility, but the VP3 peak was in complete agreement with literature. As expected, for a given concentration, the protein profiles of full and empty AAV8 did not show a significant difference. Similarly, the ssDNA assay showed consistent results when comparing the full and empty reference standards, as well as when analyzing samples of different percentages of full capsids. It must also be noted that a major benefit of running these two assays simultaneously is the ability of obtaining both the total sample concentration and the total genomic concentration of the sample, as reported in Tables 1-2.

A method according to aspects of the present disclosure offers a fast, high-throughput alternative that can be used to quickly iterate through batches of samples, and without requiring highly specialized equipment that requires significant levels of training. Within 2-3 hours, including denaturing and digestion times, depending on the number of samples being analyzed, a method according to aspects of the present disclosure estimated the percentage of full capsids with an average error rate of 16% using a total volume of less than 10 μL per sample.

Fast analysis processing time and high-throughput nature of a method according to aspects of the present disclosure provides that each additional sample analyzed only adds minimally to the total analysis time.

### Example 2

### AAV Empty/Full Analysis

5 microliters of a sample containing AAV particles is mixed with Proteinase K/urea solution in a microtiter plate and heated for 1 hour at 55° C. followed by 20 minutes at 95° C., producing extracted AAV single-stranded DNA (ssDNA).

A further 5 microliters of the sample containing AAV particles is mixed with 7 microliters of a denaturing solution in a microtiter plate and heated at 100° C. for 5 minutes to extract the VP1, VP2 and VP3 AAV capsid proteins.

The extracted AAV nucleic acids and the extracted AAV proteins are labeled and subjected to capillary electrophoresis in a microfluidic device. The detected signals from the detectable labels of the extracted AAV nucleic acids and the extracted AAV proteins are normalized to AAV reference samples and a ratio of Empty:Full AAV particles is calculated.

### Size Standard DNA Ladder Preparation

Thaw the DNA size standard (ladder) vial on ice. Spin down the ladder and heat-denature at 70° C. for 2 minutes. Immediately snap cool on ice for 5 minutes.

To prepare the ladder for use, make 1× Sample Buffer by adding 200 microliters Sample Buffer Concentrate to 1800 microliters DEPC treated or nuclease-free water.

Combine 116 microliters of 1× Sample Buffer with 4 microliters of denatured ladder. Pipette up and down carefully to mix and transfer to Ladder Tube.

Proteinase K solution Preparation

Dilute Proteinase K powder in 1.25 milliliters of diethyl pyrocarbonate (DEPC) treated or nuclease free water.

### AAV Digestion Mixture Preparation for Nucleic Acid Extraction

Prepare AAV Digestion Mixture by mixing 90 microliters of 2M Urea for every 10 microliters Proteinase K.

### Nucleic Acid Extraction

Mix 5 μL AAV sample with AAV Digestion Mixture (Proteinase K/urea solution) (5 microliters) into a PCR tube or plate. Pipette mixture up and down carefully to mix.

Mix 5 μL AAV standard with AAV Digestion Mixture (5 microliters) into a PCR tube or plate. Pipette mixture up and down carefully to mix.

Place AAV samples/Digestion mixture and standard/Digestion Mixture in a thermocycler and heat for 1 hour at 55° C. followed by 20 minutes at 95° C. producing extracted ssDNA from the samples and standard.

Centrifuge the extracted ssDNA from the samples and standard and then transfer the supernatant to a 384 well plate. Spin down the 384 well plate after transfer.

### Nucleic Acid Gel-Dye Solution Preparation

Transfer 90 microliters dye concentrate to a tube containing the gel matrix. Vortex and invert the tube several times until the resulting dye-gel mixture is well mixed and then spin it down for a few seconds. Transfer the dye-gel mixture into a spin filter and centrifuge at 9300 rcf for 10 minutes at room temperature.

### DNA Chip Preparation

Before use, rinse and completely aspirate each active well twice with nuclease-free DI water (Milli-Q® or equivalent).

Add 75 microliters gel-dye mixture to appropriate chip wells, and 120 microliters gel-dye mixture to another well.

Add 100 microliters of the DNA size standard ladder to one of the chip wells.

Following loading, capillary electrophoresis is performed wherein the material in the wells is flowed through the microchannels of a microfluidic device to a detection region.The detected signals from the detectable labels of the extracted AAV nucleic acids are normalized to AAV reference samples and a ratio of Empty:Full AAV particles is calculated.

### AAV Empty/Full Protein Gel-Dye Solution Preparation

Allow the chip and reagents to equilibrate to room temperature for at least 30 minutes before use.

To make the gel-dye solution, using a reverse pipetting technique, transfer 520 microliters gel matrix to the top basket of a provided spin filter. Transfer 20 microliters dye concentrate to the gel matrix in the spin filter. Invert, and vortex in the inverted orientation until the gel-dye is a uniform blue color. For the destain solution, transfer 250 microliters (HT) of gel matrix to a second spin filter. Centrifuge both the gel-dye and the destaining solution at 9300 rcf for 8 minutes at room temperature.

### AAV Empty/Full Protein Ladder, Buffer and Sample Preparation

Prepare the protein denaturing solution by pipetting 700 microliters of sample buffer into a 2 mL centrifuge vial, adding 24.5 microliters of 1 M DTT and mixing. Seven microliters of the protein denaturing solution is added to five microliters of the AAV sample, and to five microliters of a protein standard. Each mixture is transferred into a separate well of a microtiter plate. Twelve microliters of a protein standard ladder is deposited into a well on the plate. The samples, standards, and ladder are denatured by heating at 100° C. for 5 minutes. Next, 32 microliters of water is added to each sample well and mixed and 120 microliters of water is added to the ladder and mixed.

### AAV Empty/Full Protein Chip Preparation

75 microliters of gel-dye mixture is added to 3 wells of a microfluidic device, and 120 microliters gel-dye mixture to a separate well. Then add 75 microliters of destaining solution to each of two separate wells Further, add 120 microliters of the marker solution to a further separate well.

Following loading, capillary electrophoresis is performed wherein the material in the wells is flowed through the microchannels of a microfluidic device to a detection region. The detected signals from the detectable labels of the extracted AAV proteins are normalized to AAV reference samples and, in combination with the data derived from analysis of the extracted AAV nucleic acids and nucleic acid standards, described above, a ratio of Empty:Full AAV particles is calculated.

### AAV Empty/Full Protein Analysis

To normalize the protein concentration of the AAV sample relative to the AAV reference standard, in this example, VP3 Corrected Area inputs are used. The arrival time for VP3 peaks is ˜28-30 s under conditions used in this example. To obtain the combined corrected area:

Exclude the peak assigned to VP1 (if it is assigned)

Exclude the peak assigned to VP2 (if it is assigned)

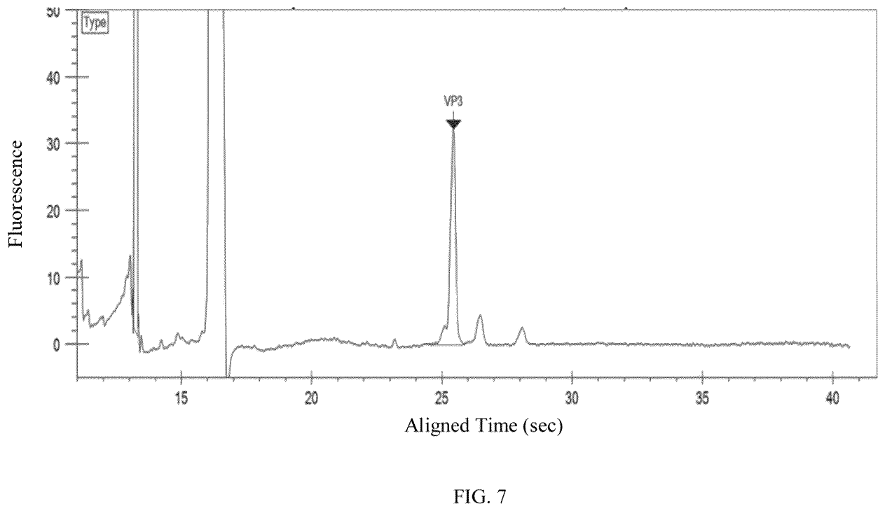

Manually extend the VP3 baseline to include all the area under the VP3 peak. The corrected area for the combined peaks is now available in the peak table on the GX Touch Reviewer. FIG. 7 is an electropherogram showing the integration of the VP3 peaks for Empty:Full ratio analysis in this example.

### AAV Empty/Full DNA Analysis

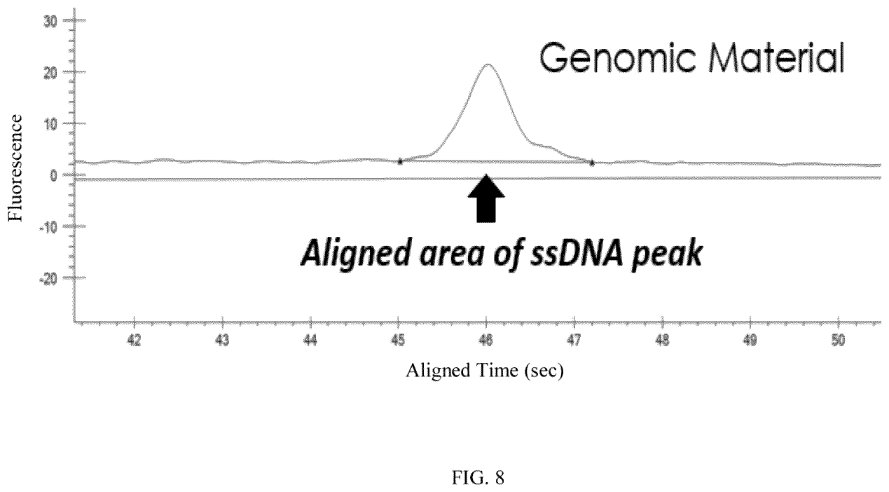

The ssDNA aligned area is used to determine the empty:full ratio of the AAV sample. The arrival time of the ssDNA peak is ˜45-47 s under conditions used in this example. The baseline of the ssDNA peak may be manually set as shown in the electropherogram showing integration of ssDNA peak of FIG. 8. The aligned area of the ssDNA peak is indicated.

AAV Empty Analysis with AAV Empty/Full Calculator

To calculate the sample % ratio of full AAV capsids to empty AAV capsids for the sample, the following values are obtained from analysis of the electropherograms generated:
1) The standard % percentage full ratio (obtained from commercially obtained AAV standard label information);
2) The measured protein content of the AAV standard (measured on plate);
3) the measured DNA content of the AAV standard (measured on plate); and
4) the measured protein and measured DNA content for each unknown AAV sample

A ratio of concentration of ssDNA between sample and standard is calculated according to equation (4).

A ratio of concentration of protein between sample and standard is calculated according to equation (3).

The fractional amount of full AAV capsids in a sample is calculated by the ratio of ssDNA and protein between sample and standard and the given percentage of full capsids in the sample is given by equation (6).

### Example 3

### Analysis of Partially Full Capsids

In this example, samples were used which contained full AAV capsids or partially full AAV capsids with viral genome content greater than 1E12 VG/mL and the method for preparation of ssDNA and virus protein described above was used with a sieving polymer matrix at 2% wt/vol with a viscosity between 40-53 centistokes (cSt).

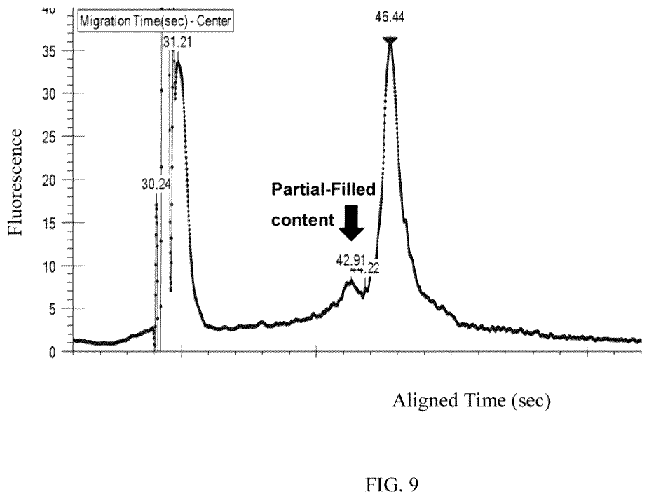

FIG. 9 is an electropherogram showing integration of an ssDNA peak for Partially Full/Full analysis of AAV nucleic acid from samples containing partially full capsids or full AAV capsids.

### Example 4

### Removal of Residual DNA/RNA Prior to Empty/Full Analysis

Some samples may contain residual DNA/RNA that is present outside of the AAV capsid, but in solution of the sample. The residual DNA/RNA can be removed by treatment with bezonase according to aspects of the present disclosure. An example procedure is:

Dilute 10 microliters of the sample to a working volume of 50 microliters to approximately with 1×10¹² genome copies/milliliter (GC/mL). PBS or PBS +0.001% Pluronic may be used as a diluent.

To 50 microliters of diluted sample, add 1 microliter of benzonase solution (≥2.5-25 units/μL)

Incubate at 37° C. for 10 minutes.

Add 1 microliter of 30 mM ETDA

Heat at 75° C. for 10 minutes

Filter the heat treated material with 100 kDa molecular weight cut-off filter, such as Amicon® Ultra-0.5 mL Centrifugal Filter, 100K. The final filtered volume is expected to be ˜15 which may then be used as the fluid sample in a method of characterizing a population of particles putatively containing recombinant nucleic acids in a fluid sample of the present disclosure.

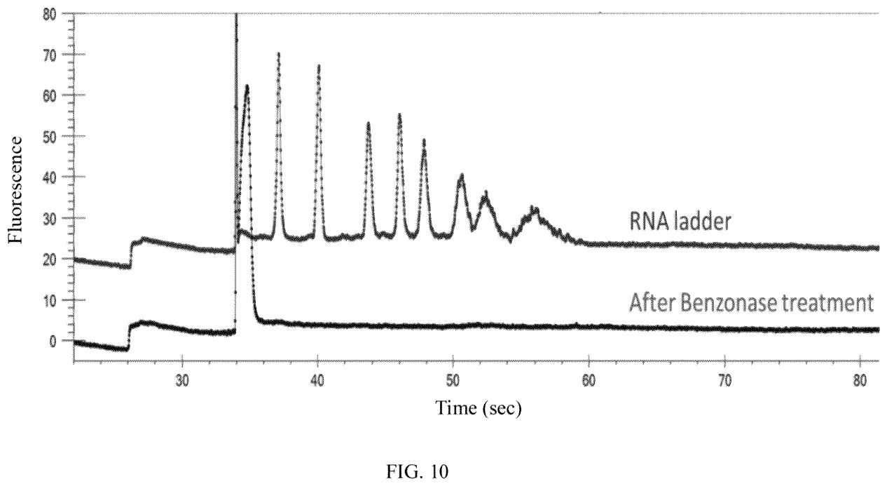

FIG. 10 is an electropherogram showing results for an RNA ladder before and after treatment with benzonase. The fragment peaks ranging in size from 200-6000 nt were reduced in size to less than 200 nt with treatment. Treatment with benzonase and filtering may be applied prior to running the AAV DNA Assay to remove residual ssDNA or RNA in the sample that is not encapsulated by AAV particles.

### Example 5

Binding of ssDNA to purification columns and recovery

Optionally, a ssDNA clean-up procedure can be used. An example procedure for AAV8 is:

Add 50 μL of AAV8 (neat or treated with proteinase k) to the QlAquick PCR column.

Incubate for 30 minutes at room temperature with binding buffer.

Wash the samples with 750 μL of purification kit wash buffer.

Elute with elution buffer that matches AAV DNA kit sample buffer (1:1 v/v of PBS and 2 M Urea)

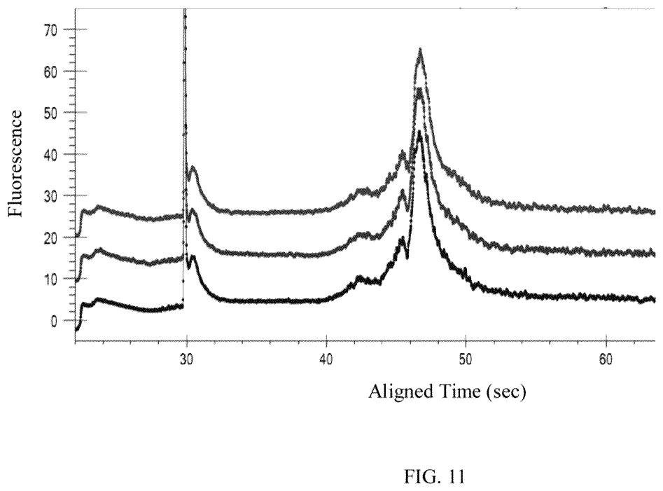

FIG. 11 shows an overlay electropherogram of 3 sips of AAV8 ssDNA obtained after recovery from binding to QlAquick PCR purification kit. The traces show AAV-DNA detection for samples that were purified using the QlAquick kit. The three traces show repeatability for peak shape and area under the curve.

### Example 6

### Analysis of Partially Full Virus Capsids

In this example, samples were used which contained full AAV capsids or partially full AAV capsids with viral genome content greater than 1E12 VG/mL and the method for preparation of ssDNA and virus protein described above was used with a sieving polymer matrix at 2% wt/vol with a viscosity between 40-53 centistokes (cSt).

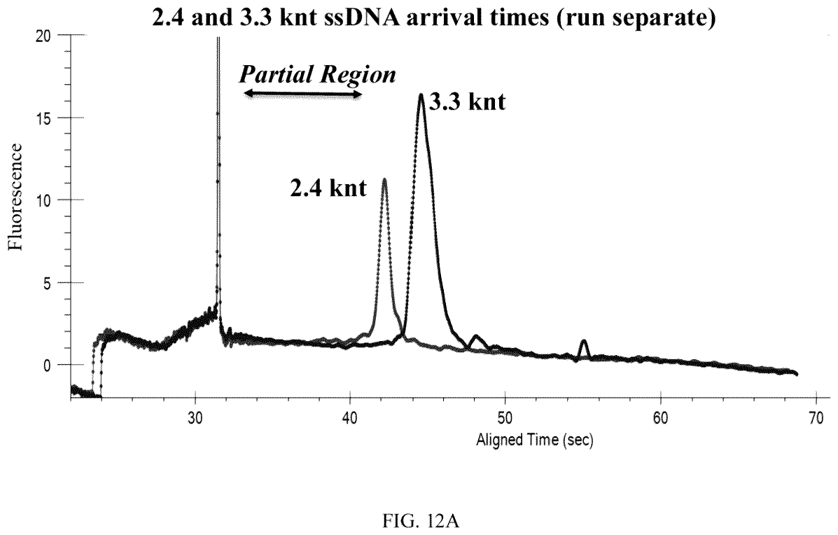

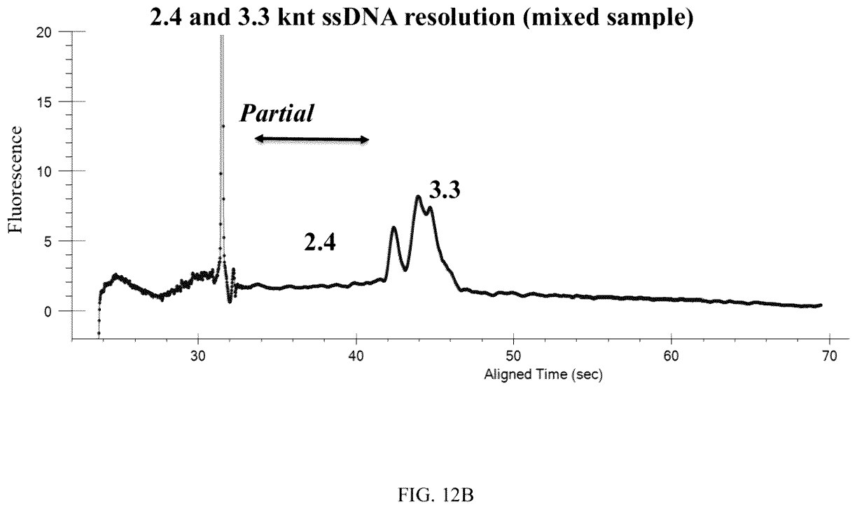

FIG. 12A is an overlay electropherogram showing detection of a 2.4 kilonucleotide (knt) SSDNA and a 3.3 knt SSDNA in separate capillary electrophoresis runs.

FIG. 12B is an electropherogram showing detection of a 2.4 kilonucleotide (knt) SSDNA and a 3.3 knt SSDNA in a single capillary electrophoresis run.

### Example 7

### Analysis of Multiple AAV Serotypes

In this example, samples were used which contained full AAV2, AAV8, or AAV9 capsids or empty AAV2, AAV8, or AAV9 capsids and the method for preparation of ssDNA and virus protein described above was used for each serotype sample. For each assay, the extracted proteins were compared to an AAV8 standard, and the extracted nucleic acids were compared to an AAV8 standard. Using AAV8 as a reference standard, the error for AAV9 and AAV2 were within the ˜12% error found for matching AAV8 standard to AAV8 samples.

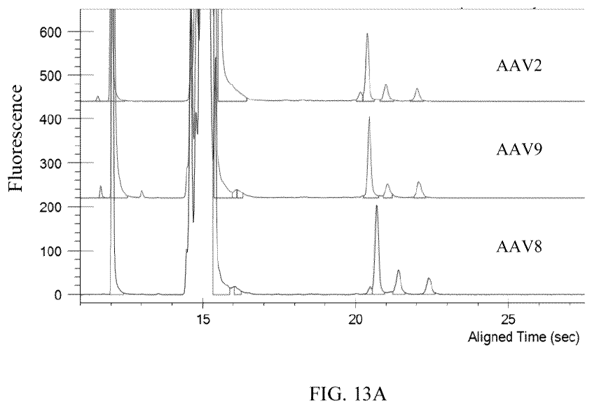

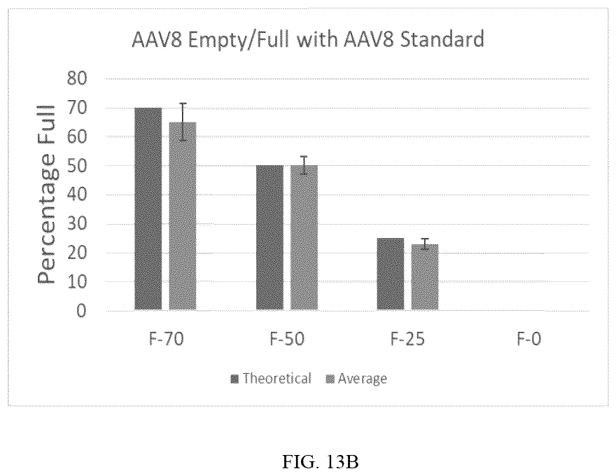

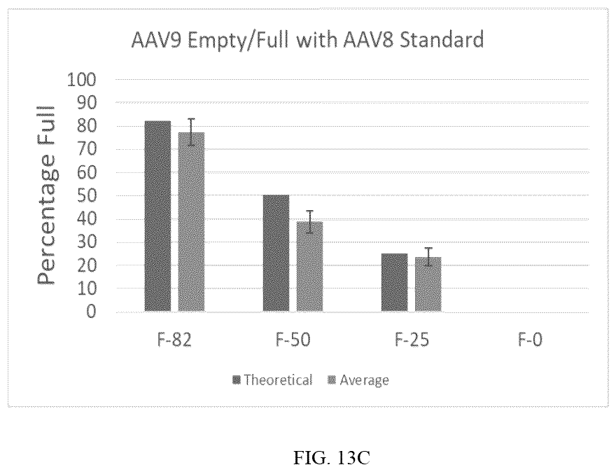

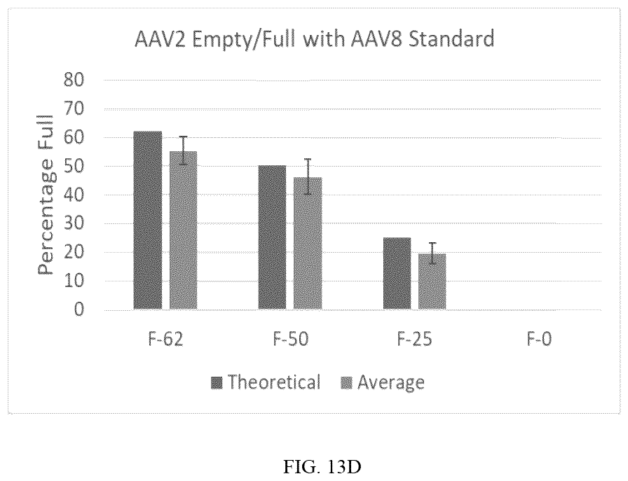

FIG. 13A is an electropherogram showing extracted viral protein assay results for each serotype indicated. Viral protein resolution and full-width at half-maximum (FWHM) is similar for AAV2, AAV8, or AAV9 virus particles.

FIG. 13B is a graph showing extracted nucleic acids assay results for 70% full AAV8 virus particles (F-70), 50% full AAV8 virus particles (F-50), 25% full AAV8 virus particles (F-25), and empty AAV8 virus particles (F-0). For each pair of bars on the graph, the left-hand bar represents the theoretical percentage full as calculated, and the right-hand bar represents the average percentage full as measured and calculated using equation (6).

FIG. 13C is a graph showing extracted nucleic acids assay results for 82% full AAV9 virus particles (F-82), 50% full AAV9 virus particles (F-50), 25% full AAV9 virus particles (F-25), and empty AAV9 virus particles (F-0). For each pair of bars on the graph, the left-hand bar represents the theoretical percentage full as calculated, and the right-hand bar represents the average percentage full as measured and calculated using equation (6).

FIG. 13D is a graph showing extracted nucleic acids assay results for 62% full AAV2 virus particles (F-62), 50% full AAV2 virus particles (F-50), 25% full AAV2 virus particles (F-25), and empty AAV2 virus particles (F-0). For each pair of bars on the graph, the left-hand bar represents the theoretical percentage full as calculated, and the right-hand bar represents the average percentage full as measured and calculated using equation (6).

Non-virus carrier particles 5 microliters of a sample containing liposomal particles putatively containing recombinant DNA or RNA is mixed with Proteinase K/urea solution in a microtiter plate and heated for 1 hour at 55° C. followed by 20 minutes at 95° C., producing extracted single-stranded DNA (ssDNA) or extracted RNA.

A further 5 microliters of the sample containing liposomal particles putatively containing recombinant DNA or RNA is mixed with 7 microliters of a denaturing solution in a microtiter plate and heated at 100° C. for 5 minutes to extract any liposomal proteins.

The extracted liposomal nucleic acids and the extracted liposomal proteins are labeled and subjected to capillary electrophoresis in a microfluidic device. The detected signals from the detectable labels of the extracted liposomal nucleic acids and the extracted liposomal proteins are normalized to reference samples and a ratio of Empty:Full liposomal particles and/or Partially Full:Full liposomal particles is calculated using equations 1, 2, 3, 4, 5, 6, 7, and 8—or any subset thereof.

### ITEMS

Item 1.) A method of characterizing a population of particles putatively containing recombinant nucleic acids in a fluid sample to determine one or more of: 1) a ratio of full particles to empty particles in the fluid sample, and 2) a ratio of full particles or empty particles to partially full particles, comprising:
extracting the recombinant nucleic acids from a first aliquot of the fluid sample containing the particles, producing extracted recombinant nucleic acids;
labeling the extracted recombinant nucleic acids, producing labeled extracted recombinant nucleic acids;
flowing the labeled extracted recombinant nucleic acids through a polymeric separation medium in a microchannel into a detection region in fluid communication with the microchannel, the detection region in signal communication with a sensor capable of detecting a signal from the detectable nucleic acid label of the labeled extracted recombinant nucleic acids, whereby the labeled extracted recombinant nucleic acids are separated according to size by flowing the labeled extracted recombinant nucleic acids through the polymeric separation medium of the microchannel;
detecting the detectable label of the labeled extracted recombinant nucleic acids in the detection region to determine: a) an amount of time taken by the labeled extracted recombinant nucleic acids to flow through the polymeric separation medium in the microchannel into the detection region, indicative of size of the recombinant nucleic acids in the fluid sample, and/or b) strength of the signal of the detectable label in the detection region, representative of the amount of labeled extracted recombinant nucleic acids present, and indicative of concentration of the labeled extracted recombinant nucleic acids in the fluid sample; and
comparing a) to a reference standard representative of full particles and, based on the comparison, determining a ratio of full particles to partially full particles, producing a first assay result and/or comparing b) to a reference standard representative of full particles and, based on the comparison, determining a ratio of full particles to empty particles in the fluid sample, producing a first assay result;
thereby characterizing the population of particles in the fluid sample.

Item 2.) The method of item 1, wherein labeling the extracted recombinant nucleic acids comprises introducing the extracted recombinant nucleic acids into a well and/or microchannel of a microfluidic device, the well and/or microchannel comprising a polymeric separation medium and a detectable nucleic acid label, whereby the detectable nucleic acid label binds to the extracted recombinant nucleic acids, producing labeled extracted recombinant nucleic acids in the well and/or microchannel.

Item 3.) The method of item 1 or 2, further comprising: determining an amount of particle protein present in the fluid sample.

Item 4.) The method of item 3, wherein the amount of particle protein in the fluid sample is determined using information about a total number of particles in the fluid sample without assaying the particle protein in the sample.

Item 5.) The method of item 3, comprising assaying particle protein in a second aliquot of the fluid sample.

Item 6.) The method of any one of items 1, 2, 3, or 5, comprising:
extracting protein from a second aliquot of the fluid sample containing the particles, producing extracted proteins of the particles;
labeling the extracted proteins of the particles, producing labeled extracted proteins of the particles;
flowing the labeled extracted proteins of the particles through the polymeric separation medium in the microchannel into a detection region in fluid communication with the microchannel, the detection region in signal communication with a sensor capable of detecting a signal from the detectable label of the labeled extracted proteins, whereby the labeled extracted proteins are separated according to size by flowing the extracted proteins through the polymeric separation medium of the microchannel;
detecting the detectable label of the labeled extracted proteins to determine: c) an amount of time taken by the labeled extracted proteins to flow through the polymeric separation medium in the microchannel into the detection region, and d) strength of the signal of the detectable label representative of the amount of labeled extracted proteins present;
comparing c) and d) to a reference standard, the reference standard representing a known amount of the protein, thereby determining an amount of protein in the sample, and thereby producing a second assay result; and
comparing the first assay result and the second assay result, thereby determining one or more of: 1) a ratio of full viral particles to empty viral particles in the fluid sample, 2) a ratio of full viral particles or empty viral particles to partially full viral particles, and 3) a ratio of viral particles containing an intact recombinant genome to viral particles containing an incomplete recombinant genome, thereby characterizing the population of recombinant viral particles in the fluid sample.

Item 7.) The method of item 6, wherein labeling the extracted proteins of the particles comprises introducing the extracted proteins of the particles into a well and/or microchannel of a microfluidic device, the well and/or microchannel comprising a polymeric separation medium and a detectable protein label, whereby the detectable protein label binds to the extracted proteins of the particles, producing labeled extracted proteins of the particles in the well and/or microchannel.

Item 8.) The method of any one of items 1 to 7, wherein the particles are recombinant virus particles having a single-stranded DNA or RNA genome in the range of 500-7000 nucleotides in length.

Item 9.) The method of any one of items 1 to 8, wherein the particles are selected from the group consisting of: recombinant adeno-associated virus (AAV) particles, recombinant retrovirus particles, recombinant lentivirus particles, and recombinant adenovirus particles.

Item 10.) The method of any one of items 1 to 9, wherein the particles are recombinant virus particles in the fluid sample present in an amount of about 1×10¹⁰ recombinant viral particles/milliliter to about 1×10¹⁴ recombinant viral particles/milliliter.

Item 11.) The method of any one of items 1 to 10, wherein extracting nucleic acids from a first aliquot of the fluid sample containing particles comprises contacting the first aliquot of the fluid sample containing particles with a proteinase and a denaturing agent.

Item 12.) The method of item 11, wherein the proteinase is a serine protease.

Item 13.) The method of item 12, wherein the proteinase is selected from the group consisting of: proteinase K, subtilisin, trypsin, chymotrypsin, thrombin, plasmin, elastase, pronase, and lactoferrin.

Item 14.) The method of item 11, wherein the denaturing agent is heat, a chaotropic agent, a detergent, or a mixture thereof.

Item 15.) A method of characterizing a population of virus particles putatively containing recombinant nucleic acids in a fluid sample to determine one or more of: 1) a ratio of full virus particles to empty virus particles in the fluid sample, 2) a ratio of full virus particles or empty virus particles to partially full virus particles, comprising:
extracting viral nucleic acids from a first aliquot of the fluid sample;
assaying the extracted viral nucleic acids by microfluidic electrophoretic assay;
calculating a ratio of nucleic acid concentration in the fluid sample to nucleic concentration in a reference standard, calculating a ratio of protein concentration in the fluid sample to protein concentration in a reference standard, and calculating a fractional amount of full capsids, (3, in the sample according to:

$$β = \frac{N ( f )}{N ( f ) + N ( e )} = \frac{{β}_{s} {R}_{DNA}}{{R}_{p}} , \qquad ( 6 )$$

wherein N is a total number of capsids, f and e refer to full and empty, respectively, βs is the percent of full capsids in the reference standard, R_{DNA} is the ratio of concentration of viral nucleic acids in the sample to the concentration of viral nucleic acids in the reference standard, and R_{P} is the ratio of concentration of viral proteins in the sample to the concentration of viral proteins in the reference standard.

Item 16.) The method of item 15, comprising: extracting viral proteins from a second aliquot of the fluid sample; and assaying at least one viral protein of the extracted viral proteins by microfluidic electrophoretic assay.

Item 17.) The method of item 15, wherein protein concentration in the fluid sample is calculated by reference to a standard and no protein assay is performed.

Item 18.) A kit for characterizing nucleic acid content of a population of particles putatively containing recombinant nucleic acids in a fluid sample, comprising: a nucleic acid label, a gel, a nucleic acid ladder standard, a nucleic acid storage buffer, a nucleic acid sample buffer, a proteinase, and a denaturing agent.

Item 19.) The kit of item 18, for characterizing protein content of the population of particles in the fluid sample, further comprising: a protein dye, a protein ladder standard, a protein storage buffer, a protein sample buffer, and a wash buffer.

Item 20.) The kit of item 18 or 19, wherein the nucleic acid label is a fluorescent nucleic acid intercalator.

Item 21.) The kit of any one of items 18 to 20, wherein the proteinase is a serine protease.

Item 22.) The kit of any one of items 18 to 21, wherein the proteinase is selected from the group consisting of: proteinase K, subtilisin, trypsin, chymotrypsin, thrombin, plasmin, elastase, pronase, and lactoferrin.

Item 23.) The kit of any of items 18 to 22, wherein the denaturing agent is a chaotropic agent, a detergent, or a mixture thereof

Item 24.) The kit of any of items 18 to 23, wherein the proteinase is proteinase K and the chaotropic agent is urea.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of characterizing a population of particles putatively containing recombinant nucleic acids in a fluid sample to determine one or more of: 1) a ratio of full particles to empty particles in the fluid sample, and 2) a ratio of full particles or empty particles to partially full particles, comprising:
extracting the recombinant nucleic acids from a first aliquot of the fluid sample containing the particles, producing extracted recombinant nucleic acids;
labeling the extracted recombinant nucleic acids, producing labeled extracted recombinant nucleic acids;
flowing the labeled extracted recombinant nucleic acids through a polymeric separation medium in a microchannel into a detection region in fluid communication with the microchannel, the detection region in signal communication with a sensor capable of detecting a signal from the detectable nucleic acid label of the labeled extracted recombinant nucleic acids, whereby the labeled extracted recombinant nucleic acids are separated according to size by flowing the labeled extracted recombinant nucleic acids through the polymeric separation medium of the microchannel;
detecting the detectable label of the labeled extracted recombinant nucleic acids in the detection region to determine: a) an amount of time taken by the labeled extracted recombinant nucleic acids to flow through the polymeric separation medium in the microchannel into the detection region, indicative of size of the recombinant nucleic acids in the fluid sample, and/or b) strength of the signal of the detectable label in the detection region, representative of the amount of labeled extracted recombinant nucleic acids present, and indicative of concentration of the labeled extracted recombinant nucleic acids in the fluid sample; and
comparing a) to a reference standard representative of full particles and, based on the comparison, determining a ratio of full particles to partially full particles, producing a first assay result and/or comparing b) to a reference standard representative of full particles and, based on the comparison, determining a ratio of full particles to empty particles in the fluid sample, producing a first assay result;
thereby characterizing the population of particles in the fluid sample.

2. The method of claim 1, wherein labeling the extracted recombinant nucleic acids comprises introducing the extracted recombinant nucleic acids into a well and/or microchannel of a microfluidic device, the well and/or microchannel comprising a polymeric separation medium and a detectable nucleic acid label, whereby the detectable nucleic acid label binds to the extracted recombinant nucleic acids, producing labeled extracted recombinant nucleic acids in the well and/or microchannel.

3. The method of claim 1, further comprising: determining an amount of particle protein present in the fluid sample.

4. The method of claim 3, wherein the amount of particle protein in the fluid sample is determined using information about a total number of particles in the fluid sample without assaying the particle protein in the sample.

5. The method of claim 3, comprising assaying particle protein in a second aliquot of the fluid sample.

6. The method of claim 1, comprising:
extracting protein from a second aliquot of the fluid sample containing the particles, producing extracted proteins of the particles;
labeling the extracted proteins of the particles, producing labeled extracted proteins of the particles;
flowing the labeled extracted proteins of the particles through the polymeric separation medium in the microchannel into a detection region in fluid communication with the microchannel, the detection region in signal communication with a sensor capable of detecting a signal from the detectable label of the labeled extracted proteins, whereby the labeled extracted proteins are separated according to size by flowing the extracted proteins through the polymeric separation medium of the microchannel;
detecting the detectable label of the labeled extracted proteins to determine: c) an amount of time taken by the labeled extracted proteins to flow through the polymeric separation medium in the microchannel into the detection region, and d) strength of the signal of the detectable label representative of the amount of labeled extracted proteins present;
comparing c) and d) to a reference standard, the reference standard representing a known amount of the protein, thereby determining an amount of protein in the sample, and thereby producing a second assay result; and
comparing the first assay result and the second assay result, thereby determining one or more of: 1) a ratio of full viral particles to empty viral particles in the fluid sample, 2) a ratio of full viral particles or empty viral particles to partially full viral particles, and 3) a ratio of viral particles containing an intact recombinant genome to viral particles containing an incomplete recombinant genome, thereby characterizing the population of recombinant viral particles in the fluid sample.

7. The method of claim 6, wherein labeling the extracted proteins of the particles comprises introducing the extracted proteins of the particles into a well and/or microchannel of a microfluidic device, the well and/or microchannel comprising a polymeric separation medium and a detectable protein label, whereby the detectable protein label binds to the extracted proteins of the particles, producing labeled extracted proteins of the particles in the well and/or microchannel.

8. The method of claim 1, wherein the particles are recombinant virus particles having a single-stranded DNA or RNA genome in the range of 500-7000 nucleotides in length.

9. The method of claim 1, wherein the particles are selected from the group consisting of: recombinant adeno-associated virus (AAV) particles, recombinant retrovirus particles, recombinant lentivirus particles, and recombinant adenovirus particles.

10. The method of claim 1, wherein the particles are recombinant virus particles in the fluid sample present in an amount of about 1×10¹⁰ recombinant viral particles/milliliter to about 1×10¹⁴ recombinant viral particles/milliliter.

11. The method of claim 1, wherein extracting nucleic acids from a first aliquot of the fluid sample containing particles comprises contacting the first aliquot of the fluid sample containing particles with a proteinase and a denaturing agent.

12.-17. (Canceled)

18. A kit for characterizing nucleic acid content of a population of particles putatively containing recombinant nucleic acids in a fluid sample, comprising:
a nucleic acid label, a gel, a nucleic acid ladder standard, a nucleic acid storage buffer, a nucleic acid sample buffer, a proteinase, and a denaturing agent.

19. The kit of claim 18, for characterizing protein content of the population of particles in the fluid sample, further comprising:
a protein dye, a protein ladder standard, a protein storage buffer, a protein sample buffer, and a wash buffer.

20.The kit of claim 18, wherein the nucleic acid label is a fluorescent nucleic acid intercalator.

21. The kit of claim 18, wherein the proteinase is a serine protease.

22. The kit of claim 18, wherein the proteinase is selected from the group consisting of: proteinase K, subtilisin, trypsin, chymotrypsin, thrombin, plasmin, elastase, pronase, and lactoferrin.

23. The kit of claim 18, wherein the denaturing agent is a chaotropic agent, a detergent, or a mixture thereof.

24. The kit of claim 18, wherein the proteinase is proteinase K and the chaotropic agent is urea.

25. The kit of claim 18, further comprising a nucleic acid AAV standard.

26. The kit of claim 18, further comprising a protein AAV standard.
